SEQUENCE LISTING (20 sequences)

SEQ ID NO: 1 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 2 - protein, 699 aa, Homo sapiens
YDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 3 - protein, 699 aa, Homo sapiens
YDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 4 - protein, 699 aa, Homo sapiens
YDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 5 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLMSDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQEWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTVMSKLWFKQDDKKKKPKACLNFEFFSPFAYVDPLHCNMAYLYLELLKDSLNEYAYAAELAGLSYDLQNTIYGMYLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQSLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 6 - protein, 1019 aa, Homo sapiens
MRYRLAWLLHPALPSTFRSVLGARLPPPERLCGFQKKTYSKMNNPAIKRIGNHITKSPEDKREYRGLELANGIKVLLISDPTTDKSSAALDVHIGSLSDPPNIAGLSHFCEHMLFLGTKKYPKENEYSQFLSEHAGSSNAFTSGEHTNYYFDVSHEHLEGALDRFAQFFLCPLFDESCKDREVNAVDSEHEKNVMNDAWRLFQLEKATGNPKHPFSKFGTGNKYTLETRPNQEGIDVRQELLKFHSAYYSSNLMAVCVLGRESLDDLTNLVVKLFSEVENKNVPLPEFPEHPFQEEHLKQLYKIVPIKDIRNLYVTFPIPDLQKYYKSNPGHYLGHLIGHEGPGSLLSELKSKGWVNTLVGGQKEGARGFMFFIINVDLTEEGLLHVEDIILHMFQYIQKLRAEGPQGWVFQECKDLNAVAFRFKDKERPRGYTSKIAGILHYYPLEEVLTAEYLLEEFRPDLIEMVLDKLRPENVRVAIVSKSFEGKTDRTEEWYGTQYKQEAIPDEVIKKWQNADLNGKFKLPTKNEFIPTNFEILPLEKEATPYPALIKDTAMSKLWFKQDDKFFLPKACLNFEFFSRYIYADPLHCNMTYLFIRLLKDDLKEYTYAARLSGLSYGIASGMNAILLSVKGYNDKQPILLKKIIEKMATFEIDEKRFEIIKEAYMRSLNNFRAEQPHQHAMYYLRLLMTEVAWTKDELKEALDDVTLPRLKAFIPQLLSRLHIEALLHGNITKQAALGIMQMVEDTLIEHAHTKPLLPSQLVRYREVQLPDRGWFVYQQRNEVHNNCGIEIYYQTDMQSTSENMFLELFCQIISEPCFNTLRTKEQLGYIVFSGPRRANGIQGLRFIIQSEKPPHYLESRVEAFLITMEKSIEDMTEEAFQKHIQALAIRRLDKPKKLSAECAKYWGEIISQQYNFDRDNTEVAYLKTLTKEDIIKFYKEMLAVDAPRRHKVSVHVLAREMDSCPVVGEFPCQNDINLSQAPALPQPEVIQNMTEFKRGLPLFPLVKPHINFMAAKL

SEQ ID NO: 7 - protein, 681 aa, Homo sapiens
QYQTRSPSVCLSEACVSVTSSILSSMDPTVDPCHDFFSYACGGWIKANPVPDGHSRWGTFSNLWEHNQAIIKHLLENSTASVSEAERKAQVYYRACMNETRIEELRAKPLMELIERLGGWNITGPWAKDNFQDTLQVVTAHYRTSPFFSVYVSADSKNSNSNVIQVDQSGLGLPSRDYYLNKTENEKVLTGYLNYMVQLGKLLGGGDEEAIRPQMQQILDFETALANITIPQEKRRDEELIYHKVTAAELQTLAPAINWLPFLNTIFYPVEINESEPIVVYDKEYLEQISTLINTTDRCLLNNYMIWNLVRKTSSFLDQRFQDADEKFMEVMYGTKKTCLPRWKFCVSDTENNLGFALGPMFVKATFAEDSKSIATEIILEIKKAFEESLSTLKWMDEETRKSAKEKADAIYNMIGYPNFIMDPKELDKVFNDYTAVPDLYFENAMRFFNFSWRVTADQLRKAPNRDQWSMTPPMVNAYYSPTKNEIVFPAGILQAPFYTRSSPKALNFGGIGVVVGHELTHAFDDQGREYDKDGNLRPWWKNSSVEAFKRQTECMVEQYSNYSVNGEPVNGRHTLGENIADNGGLKAAYRAYQNWVKKNGAEHSLPTLGLTNNQLFFLGFAQVWCSVRTPESSHEGLITDPHSPSRFRVIGSLSNSKEFSEHFRCPPGSPMNPPHKCEVW

SEQ ID NO: 8 - protein, 21 aa, artificial sequence
  synthetic peptide
METDTLLLWVLLLWVPGSTGD

SEQ ID NO: 9 - protein, 12 aa, Homo sapiens
ERKCCVECPPCP

SEQ ID NO: 10 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 11 - protein, 927 aa, artificial sequence
  synthetic polypeptide
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 12 - protein, 1247 aa, artificial sequence [sequence omitted]

SEQ ID NO: 13 - protein, 909 aa, artificial sequence [sequence omitted]

SEQ ID NO: 14 - protein, 947 aa, Mus musculus
METDTLLLWVLLLWVPGSTGDVPRDCGCKPCICTVPPVSSVFIFPPKPKDVLTITLTPKVTCVVVAISKDDPEVQFSWFVDDVEVHTAQTQPREEQFASTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGKYDDGICKSSDCIKSAARLIQNMDASVEPCTDFFKYACGGWLKRNVIPETSSRYSNFDILRDELEVILKDVLQEPKTEDIVAVQKAKTLYRSCINESAIDSRGGQPLLKLLPDIYGWPVASDNWDQTYGTSWTAEKSIAQLNSKYGKKVLINFFVGTDDKNSTQHIIHFDQPRLGLPSRDYYECTGIYKEACTAYVDFMISVARLIRQEQSLPIDENQLSLEMNKVMELEKEIANATTKPEDRNDPMLLYNKMTLAKLQNNFSLEVNGKSFSWSNFTNEIMSTVNINIQNEEEVVVYAPEYLTKLKPILTKYSPRDLQNLMSWRFIMDLVSSLSRNYKESRNAFRKALYGTTSETATWRRCANYVNGNMENAVGRLYVEAAFAGESKHVVEDLIAQIREVFIQTLDDLTWMDAETKKKAEEKALAIKERIGYPDDIISNENKLNNEYLELNYREDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSANNFKDQSQCMVYQYGNFSWDLAGGQHLNGINTLGENIADNGGIGQAYRAYQNYVKKNGEEKLLPGLDLNHKQLFFLNFAQVWCGTYRPEYAVNSIKTDVHSPGNFRIIGTLQNSAEFADAFHCRKNSYMNPERKCRVW

SEQ ID NO: 15 - protein, 38 aa, homo sapiens
DAEFRHDSGYEVHHQKLVFFAEDVGSNKGAIIGLMVGG

SEQ ID NO: 16 - protein, 39 aa, Homo sapiens
DAEFRHDSGYEVHHQKLVFFAEDVGSNKGAIIGLMVGGV

SEQ ID NO: 17 - protein, 40 aa, Homo sapiens
DAEFRHDSGYEVHHQKLVFFAEDVGSNKGAIIGLMVGGVV

SEQ ID NO: 18 - protein, 41 aa, Homo sapiens
DAEFRHDSGYEVHHQKLVFFAEDVGSNKGAIIGLMVGGVVI

SEQ ID NO: 19 - protein, 42 aa, Homo sapiens [sequence omitted]

SEQ ID NO: 20 - protein, 43 aa, Homo sapiens
DAEFRHDSGYEVHHQKLVFFAEDVGSNKGAIIGLMVGGVVIAT

(19) United States
(12) Patent Application Publication
(10) Pub. No.: US 2008/0242590 A1
(43) Pub. Date: Oct. 2, 2008

(54) New Method 706

(75) Inventors: Christin Andersson, Sodertalje (SE); Per-Ola Freskgard, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/057,464

(22) Filed: Mar. 28, 2008

Related U.S. Application Data
(60) Provisional application No. 60/908,471, filed on Mar. 28, 2007.

(51) Int. Cl. A61K 38/02; C07K 2/00; C12N 9/50; A61P 25/28; C07K 19/00

(52) U.S. Cl. 514/2; 530/300; 435/219; 530/387.1

(57) ABSTRACT

The present invention relates fusion proteins and their use in enzymatic treatment of Alzheimer's disease patients. Said fusion protein has the formula M-A, capable of degrading amyloid beta peptide at one or more cleavage sites in its amino acid sequence, wherein M is a protein component that prolongs the half-life of the fusion protein, and A is a protein component that cleaves the amyloid beta peptide.

The present invention relates fusion proteins and their use in enzymatic treatment of Alzheimer's disease patients. Said fusion protein comprises a component that cleaves the amyloid beta (Aβ) peptide, another component that modulates the half-life in plasma; and optionally, a third component that connects the first two components.

### BACKGROUND OF THE INVENTION

The present invention relates to methods of preventing amyloid plaque formation and/or growth by reacting amyloid peptides with an enzyme that specifically recognizes amyloid peptides, and inactivates them through degradation or modification. The present invention in further relates to a method of treating Alzheimer's disease by administering an optimized amyloid peptide-degrading enzyme with improved catalytic activity and/or selectivity and also prolonged activity in blood plasma. The present invention also relates to the field of medical therapy, in particular to the field of neurodegenerative disease and provides methods of eliciting clearance mechanisms for brain amyloid in patients suffering from neurodegenerative diseases, in particular Alzheimer's disease. Furthermore, this invention relates to the use of proteins and peptides effective in eliciting such mechanisms.

The present invention describes how an Aβ-peptide degrading molecule can become a therapeutic relevant agent by attaching a molecule that modulates the stability and half-life in blood plasma. The Aβ-peptide degrading molecules described in this invention overall possesses too short plasma half-life to be useful as an effective therapeutic agent. However, by combining these degrading molecules with the described and exemplified modulator molecules in this invention, functional agents is produced that can be used effectively in treating Alzheimer's disease by administering these optimized amyloid peptide-degrading enzyme fusion proteins.

Neurodegenerative diseases, in particular Alzheimer's disease (AD), have a strong debilitating impact on a patient's life. Furthermore, these diseases constitute an enormous health, social and economic burden. AD is the most common age-related neurodegenerative condition affecting about 10% of the population over 65 years of age and up to 45% over age 85 (Vickers et al., Progress in Neurobiology 2000, 60:139-165). Presently, this amounts to an estimated 12 million cases in the US, Europe, and Japan. This situation will inevitably, worsen with the demographic increase in the number of old people in developed countries. The neuropathological hallmarks that occur in the brain of individuals suffering from AD are senile plaques and profound cytoskeletal changes coinciding with the appearance of abnormal filamentous structures and the formation of neurofibrillary tangles. Both familial and sporadic cases share the deposition in brain of extracellular, fibrillary β-amyloid as a common pathological hallmark that is believed to be associated with impairment of neuronal functions and neuronal loss (Younkin S. G., Ann. Neurol. 37, 287-288, 1995; Selkoe, D. J., Nature 399, A23-A31, 1999; Borchelt D. R. et al., Neuron 17, 1005-1013, 1996). B-amyloid deposits are composed of several species of amyloid-β peptides (Aβ); especially Aβ₄₂ is deposited progressively in amyloid plaques. AD is a progressive disease that is associated with early deficits in memory formation and ultimately leads to the complete erosion of higher cognitive function. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10:184-192).

Genetic evidence suggests that increased amounts of Aβ₄₂ are produced in many, if not all, genetic conditions that cause familial AD (Borchelt D. R. et al., Neuron 17, 1005-1013, 1996; Duff K. et al., Nature 383, 710-713, 1996; Scheuner D. et al., Nat. Med. 2, 864-870, 1996; Citron M. et al., Neurobiol. Dis. 5, 107-116, 1998), pointing to the possibility that amyloid formation may be caused either by increased generation of Aβ₄₂, or decreased degradation, or both (Glabe, C., Nat. Med. 6, 133-134, 2000). Although these are rare examples of early-onset AD, which have been attributed to genetic defects in the genes for APP, presenilin-1, and presenilin-2, the prevalent form of late-onset sporadic AD is of hitherto unknown etiologic origin. However, several risk factors have been identified that predispose an individual to develop AD, among them most prominently the epsilon4 allele of apolipoprotein E (ApoE) and the B-allele of cystatin C. The late onset and complex pathogenesis of neurodegenerative disorders pose a formidable challenge to the development of therapeutic agents.

Currently, there is no cure for AD, nor even a method to diagnose AD ante-mortem with high probability. However, β-amyloid has become a major target for the development of drugs designed to reduce its formation (Vassar, R. et al., Science 286, 735-41, 1999), or to activate mechanisms that accelerate its clearance from brain.

However, first experimental results by Schenk et al. (Nature, vol. 400, 173-177, 1999; Arch. Neurol., vol. 57, 934-936, 2000) suggest possible new treatment strategies for AD. The PDAPP transgenic mouse, which overexpresses mutant human APP (in which the amino acid at position 717 is phenylalanine instead of the normal valine), progressively develops many of the neuropathological hallmarks of AD in an age- and brain region-dependent manner. Transgenic animals were immunised with Aβ₄₂ either before the onset of AD-type neuropathologies (at 6 weeks of age) or at an older age (11 months), when amyloid-β deposition and several of the subsequent neuropathological changes were well established. Immunisation of the young animals essentially prevented the development of β-amyloid-plaque formation, neuritic dystrophy and astrogliosis. Treatment of the older animals also markedly reduced the extent and progression of these AD-like neuropathologies. It was shown that Aβ₄₂ immunisation results in the generation of anti-Aβ antibodies and that Aβ-immunoreactive monocytic/microglial cells appear in the region of remaining plaques. However, an active immunisation approach can entail serious side effects and hitherto unknown complications in human subjects.

Bard et al. (Nature Medicine, Vol. 6, Number 8, 916-919, 2000) reports that peripheral administration of antibodies against amyloid β-peptide is sufficient to reduce amyloid burden. Despite their relatively modest serum levels, the passively administered antibodies were able to cross the blood-brain barrier and enter the central nervous system, decorate plaques and induce clearance of pre-existing amyloid. However, even a passive immunisation against β-peptide may cause undesirable side effects in human patients.

The present invention is directed to using recombinant protein to treat Alzheimer's patients. The balance between the anabolic and catabolic pathways in the metabolism of the Aβ peptides is delicate. Although considerable effort has focused on the generation of the Aβ peptides, until recently considerably less emphasis has been placed on the clearance of these peptides. Removal of extracellular Aβ peptide appears to proceed through two general mechanisms; cellular internalization and extracellular degradation. The present invention describes a novel approach which will complement the natural catabolic process of amyloid β peptide.

DeMattos (PNAS 98: 8850-8855. 2001) have described the sink hypothesis that state that Aβ-peptide can be removed from CNS indirectly by lowering the concentration of the peptide in the plasma. They used an antibody that binds the Aβ-peptide in the plasma and thereby sequester Aβ from the CNS. This is accomplished because the antibody prevent influx of Aβ from the plasma to CNS and/or change the equilibrium between the plasma and CNS due to a lowering of the free Aβ concentration in plasma. Amyloid binding agents unrelated to antibodies have also been shown to be effective in removing amyloid β-peptide from CNS through the binding in plasma. Matsuoka et al. (J. Neuroscience, Vol. 23: 29-33, 2003) have presented data using two amyloid β-peptide binding agents, gelsolin and GM1, which sequester plasma Aβ and thereby reduce or prevent brain amyloidosis.

Another approach to remove or eliminate Aβ-peptide is the use of a degradation enzyme that degrades the amyloid β peptide into smaller fragments with no or lower toxicological effects which are more prone for clearance. This enzymatic digestion of the Aβ-peptide will also work through the sink hypothesis mechanism by lowering the free concentration of amyloid β peptide in plasma. However, there is also a possibility for direct clearance of amyloid β peptide in the CNS and/or CSF. This approach will not only lower the free concentration of Aβ but also directly clear the environment from the full-length peptide. This approach is advantageous because it will not increase the total (free and bound) concentration of Aβ in the plasma as been seen in cases when using amyloid β peptide binding agents such as antibodies. There are enzymes described in the literature that degrade the Aβ-peptide at multiple sites, for example NEP (Leissring et al., JBC. 278: 37314-37320, 2003). Degradation of the Aβ-peptide at multiple site will generate small fragment that are cleared from the blood stream easily.

### BRIEF DESCRIPTION OF THE DRAWINGS

FIG. 1

Degradation of amyloid β1-40 peptide (final concentration 300 nM) by commercial Neprilysin (2.4 μg/ml) or Fc-Neprilysin fusion protein (2.4 μg/ml) in buffer.

FIG. 2

Aβ40 degradation by His-Fc-Nep (SPL061128) and Neprilysin (R&D systems) in guinea pig plasma. Two concentrations of His-Fc-Nep are used, and Aβ40 levels are measured after 4 hours. Commercial Neprilysin is used as positive control, and phosphoramidon is used as Neprilysin-specific inhibitor.

FIG. 3

Aβ42 degradation by His-Fc-Nep (SPL061128) and Neprilysin (R&D systems) in guinea pig plasma. Two concentrations of His-Fc-Nep are used, and Aβ42 levels are measured after 4 hours. Commercial Neprilysin is used as positive control, and phosphoramidon is used as Neprilysin-specific inhibitor.

FIG. 4

Aβ40 degradation by His-Fc-Nep (SPL061128) and Neprilysin (R&D systems) in human plasma. Two concentrations of His-Fc-Nep are used, and Aβ40 levels are measured after 4 hours. Commercial Neprilysin is used as positive control, and phosphoramidon is used as Neprilysin-specific inhibitor.

FIG. 5

The PK profile (plasma concentration over time) for Fc-Nep fusion protein compared to commercial Neprilysin. Mice were administered with 1 mg/kg commercial Neprilysin or 1 alternatively 5 mg/kg in-house produced Fc-Nep.

FIG. 6

Enzymatic activity in cell media from expression of Fc-Neprilysin (N-terminal fusion of Fc) compared to Neprilysin-Fc (C-terminal fusion of Fc). Description: PCEP4GW-Nep-Fc: Neprilysin-Fc expressed from pCEP4 plasmid; PEAK10GW-Nep-Fc: Neprilysin-Fc expressed from pEAK10 plasmid; com.Nep: Positive control, commercially available Neprilysin; PCEP4GW-Fc-Nep: Fc-Neprilysin expressed from pCEP4 plasmid; PEAK10GW-Fc-Nep: Fc-Neprilysin expressed from pEAK10 plasmid.

FIG. 7

Soluble Aβ40 levels in plasma of female APP_{SWE}-tg mice after an acute treatment with Fc-Nep as well as treatment with the positive control, γ-secretase inhibitor M550426.

FIG. 8

Soluble Aβ42 levels in plasma of female APP_{SWE}-tg mice after an acute treatment with Fc-Nep as well as treatment with the positive control, γ-secretase inhibitor M550426.

FIG. 9

Enzymatic activity of purified protein Fc-Neprilysin (N-terminal fusion of Fc) compared to Neprilysin-Fc (C-terminal fusion of Fc).

Description: Nep-Fc: Neprilysin fused to Fc in C-terminal part of Neprilysin; Fc-Nep: Neprilysin fused to Fc in N-terminal part of Neprilysin.

FIG. 10

Mouse Aβ40 levels in plasma of female C57BL/6 mice after an acute treatment with hFc-Nep as well as treatment with the positive control, γ-secretase inhibitor M550426.

FIG. 11

Aβ40 levels in plasma at different time points after a single injection of hFc-Nep to female C57BL6 mice. The percentage shows the reduction compared to vehicle. The exposure of hFc-Nep is shown over each treatment bar in the diagram. The effect of treatment with the positive control, γ secretase inhibitor M550426 is shown in red. The LOQ line shows the limit of quantification in the assay.

FIG. 12

Mean Aβ40 (A) and Aβ42 levels (B) in plasma at different time points (from 1.5 up to 336 hours) after a single injection of mFc-Nep to female APP_{SWE}-transgenic mice. The percentage shows the reduction compared to vehicle. The table (C) shows the plasma exposure for respective groups. The effect of treatment with the positive control, γ secretase inhibitor M550426 is shown in red. The LOQ bar shows the limit of quantification in the assay. Data was analysed using two-sided t-tests in an ANOVA model with time and dose as fixed factors (*p<0.05; **p<0.01 and ***p<0.001 and n.s. non-significant).

FIG. 13

Pharmacokinetic and pharmacodynamic diagrams showing the plasma efficacy effects of Aβ40 and Aβ42, respectively, as percentage of vehicle for all time point (1.5-336 hours), as well as corresponding plasma exposure of mFc-Nep. The line in respective diagram shows the predicted exposure and effect.

In C57BL/6 mice, mFc-Nep significantly reduce mouse Aβ40 in plasma in at both 5 and 25 mg/kg at all time points (1.5, 168 and 336 hours) (FIG. 14). At 168 and 336 hours, both 5 and 25 mg/kg was analysed and the Aβ40 effects are shown to be dose-dependent. After 2 weeks, a single injection (336 hours) of 25 mg/kg mFc-Nep, significantly reduce the mouse Aβ40 levels in plasma by 73% compared to vehicle. The plasma exposure at this time point was 48 μg/ml and mFc-Nep thereby show to have a long plasma half-life.

FIG. 14

Mean Aβ40 levels in plasma at different time points (1.5, 168 and 336 hours) after a single intravenous injection of mFc-Nep to female C57BL6 mice. The percentage shows the reduction compared to vehicle. The table on the right shows the plasma exposure for respective groups. The effect of treatment with the positive control, γ secretase inhibitor M550426 is shown in red. The LOQ bar shows the limit of quantification in the assay. Data was analysed using two-sided t-tests in an ANOVA model with time and dose as fixed factors (*p<0.05; **p<0.01, ***p<0.001 and n.s. non-significant).

FIG. 15

The PK profile (plasma concentration over time) for Fc-Nep fusion protein compared to in-house produced Neprilysin. Mice were administered with a single i.v. dose of 10 or 50 nmol enzyme/kg body weight neprilysin (Nep) or Fc-Nep (1 and 5 mg/kg) to mice.

FIG. 16

Table describing degradation of amyloid β peptide 1-40 or 1-42 in human plasma or APP_{swe}-tg mouse plasma by human or mouse Fc-Neprilysin. EC₅₀ (μM) of degradation and % degradation at highest (100 μg/mL) concentration of human or mouse Fc-Neprilysin. The results are based on 2-3 independent experiments.

### DISCLOSURE OF THE INVENTION

The object of the present invention is to provide fusion proteins capable of degrading Aβ peptide. Accordingly, the present invention provides a fusion protein having the formula M-A, capable of degrading amyloid beta peptide at one or more cleavage sites in said amyloid beta peptide amino acid sequence, wherein M is a protein component that prolongs the half-life of the fusion protein, and A is a protein component that cleaves the amyloid beta peptide, wherein said M protein component is covalently connected to the N-terminus part of the A protein component.

In one aspect of the present invention, there is provided a fusion protein, wherein A is a protease.

In another aspect of the present invention, there is provided a fusion protein, wherein A is human Neprilysin.

In another aspect of the present invention, there is provided a fusion protein, wherein A is human Neprilysin, wherein said Neprilysin is extracellular Neprilysin.

In another aspect of the present invention, there is provided a fusion protein, wherein A is extracellular Neprilysin, comprising an amino acid sequence according to any one of SEQ ID NO. 1, 2, 3 or 4.

In another aspect of the present invention, there is provided a fusion protein, wherein A is insulin-degrading enzyme.

In another aspect of the present invention, there is provided a fusion protein, wherein A is endothelin-converting enzyme 1.

In another aspect of the present invention, there is provided a fusion protein, wherein A is a scaffold protein.

In another aspect of the present invention, there is provided a fusion protein, wherein M is an Fc part of an antibody. In one embodiment of this aspect, said antibody is an IgG antibody. In another embodiment of this aspect, said antibody is an IgG2 antibody.

In another aspect of the present invention, there is provided a fusion protein, wherein M is an Fc part from an IgG2 antibody and A is extracellular Neprilysin.

In another aspect of the present invention, there is provided a fusion protein, comprising an amino acid sequence according to SEQ ID NO. 11.

In another aspect of the present invention, there is provided a fusion protein, wherein M is an Fc part from an IgG2 antibody and A is insulin-degrading enzyme.

In another aspect of the present invention, there is provided a fusion protein, comprising an amino acid sequence according to SEQ ID NO. 12.

In another aspect of the present invention, there is provided a fusion protein, wherein M is an Fc part from an IgG2 antibody and A is endothelin-converting enzyme 1.

In another aspect of the present invention, there is provided a fusion protein, comprising an amino acid sequence according to SEQ ID NO. 13.

In another aspect of the present invention, there is provided a fusion protein, wherein M is selected from pegylation and glycosylation.

In another aspect of the present invention, there is provided a fusion protein, wherein M is a HSA.

In another aspect of the present invention, there is provided a fusion protein, wherein M is a HSA binding domain.

In another aspect of the present invention, there is provided a fusion protein, wherein M is a antibody binding domain.

In another aspect of the present invention, there is provided a fusion protein, wherein M and A is linked together with a linker, L.

In another aspect of the present invention, there is provided a fusion protein, wherein L is selected from a peptide and a chemical linker.

In another aspect of the present invention, there is provided a method for reducing amyloid β peptide concentration, said method comprising administration of a fusion protein, according to the invention. In one embodiment of this aspect, said reduction of amyloid β peptide is accomplished in plasma. In another embodiment of this aspect, said reduction of amyloid β peptide is accomplished in CSF. In yet another embodiment of this aspect, said reduction of amyloid β peptide is accomplished in CNS.

In another aspect of the present invention, there is provided a pharmaceutical composition capable of degrading amyloid β peptide, comprising a pharmaceutically acceptable amount of fusion protein according to the invention together with a pharmaceutically acceptable carrier or excipient.

In another aspect of the present invention, there is provided a method of prevention and/or treatment of a condition wherein of degradation of amyloid β peptide is beneficial, comprising administering to a mammal, including man in need of such prevention and/or treatment, a therapeutically effective amount of a fusion protein according to the invention.

In another aspect of the present invention, there is provided a method of prevention and/or treatment of Alzheimer's disease, systemic amyloidosis or cerebral amyloid angiopathy, comprising administering to a mammal, including man in need of such prevention and/or treatment, a therapeutically effective amount of a fusion protein according to the invention.

In another aspect of the present invention, there is provided a fusion protein according to the invention for use in medical therapy.

In another aspect of the present invention, there is provided use of a fusion protein of the invention, in the manufacture of a medicament for prevention and/or treatment of conditions wherein of degradation of amyloid β peptide is beneficial.

In another aspect of the present invention, there is provided use of a fusion protein of the invention, in the manufacture of a medicament for prevention and/or treatment of Alzheimer's disease, systemic amyloidosis or cerebral amyloid angiopathy. In one embodiment of this aspect, said medicament reduces amyloid β peptide concentration. Said reduction of amyloid β peptide is accomplished in plasma, CSF and/or CNS.

The terms used throughout this specification are defined as follows, unless otherwise limited in specific instances.

The term “modulator” refers to a molecule that prevents degradation and/or increases plasma half-life, reduces toxicity, reduces immunogenicity, or increases biological activity of a therapeutic protein. Exemplary modulators include an Fc domain as well as a linear polymer (e.g., polyethylene glycol (PEG), polylysine, dextran, etc.); a branched-chain polymer (see, for example, U.S. Pat. No. 4,289,872, U.S. Pat. No. 5,229,490; WO 93/21259); a lipid; a cholesterol group (such as a steroid); a carbohydrate or oligosaccharide; or any natural or synthetic protein, polypeptide or peptide that binds to a salvage receptor. Glycosylation is also an example of modulator that through the increase in size of the fusion protein can prolong the plasma half-life, mainly due to a change in the clearance mechanism. A modulator can also include human serum albumin (HSA) binding components which thereby prolong the plasma half-life of the fusion protein.

The term “protein” or “protein component” refers to a molecule that possesses a catalytic activity, which degrades the amyloid β peptide by protolytic cleavage at any possible site in the amino acid sequence. Examples of proteins include the neprilysin enzyme as well as other catalytic active enzymes that degrade the amyloid β peptide. Catalytic antibodies could also be used as the protein part. The protein can be a natural occurring variant from any species (e.g. human, monkey, mice) or a designed variant using rational design or molecular evolution technologies. The protein molecule can also be different polymorphic or splice variants. The protein molecule can also be an improved variant of a natural occurring variant from any species. Especially a protein can be an improved variant of neprilysin that has been modified in the structure by amino acid replacement to attain improved properties such as increased activity, improved selectivity towards the amyloid beta peptide and prolonged activity in blood plasma due to increased stability and/or reduced inhibition.

The term “fusion” refers to a molecule that is composed of a modulator molecule and a protein molecule. The modulator may be covalently linked to the protein part to create the fusion protein. A non-covalent approach can also be used to connect the protein to the modulator part.

The term “degrade”, “degrading” or “degradation” refers to a process where one starting molecule is divided in two or more molecule(s). More specifically, the amyloid β peptide (in any size from amino acid 1-43 and smaller) is cleaved to generate smaller fragments compared to the starting molecule. The cleavage can be accomplished through hydrolysis of peptide bonds or other type of reaction, which split the molecule in smaller parts.

The term “native Fc” refers to molecule or sequence comprising the sequence of a non-antigen-binding fragment resulting from digestion of whole antibody, whether in monomeric or multimeric form. The original immunoglobulin source of the native Fc may be of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. Publications WO 97/34631 and WO 96/32478 describe exemplary Fc variants, as well as interaction with the salvage receptor, and are hereby incorporated by reference. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The term “pharmacologically active” means that a substance so described is determined to have activity that affects a medical parameter (e.g., blood pressure, blood cell count, cholesterol level) or disease state (e.g., cancer, autoimmune disorders, dementia).

The term “amyloid beta peptide”, “Aβ peptide” or “amyloid β peptide” means any form of the peptide that correlate to amino acid sequence (one letter code) DAEFRHDSG YEVHHQKLVF FAEDVGSNKG AIIGLMVGGV VIAT in the human Aβ A4 protein [Precursor], corresponding to amino acid 672 to 714 in the sequence (amino acid 1-43). It also includes any shorter forms of this peptide, such as 1-38, 1-40 and 1-42 but not restricted to these forms. Moreover, Amyloid β peptide has several natural occurring forms. The human forms of Amyloid β peptide are referred to as Aβ39, Aβ40, Aβ41, Aβ42 and Aβ43. The sequences of these peptides and their relationship to the APP precursor are illustrated by FIG. 1 of Hardy et al., TINS 20, 155-158 (1997). For example, Aβ42 has the sequence:

H2N-Asp-Ala-Glu-Phe-Arg-His-Asp-Ser-Gly-Tyr-Glu-Val-His-His-Gln-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-Ile-Ala-OH. Aβ41, Aβ40 and Aβ39 differ from Aβ42 by the omission of Ala, Ala-Ile, and Ala-Ile-Val respectively from the C-terminal end. Aβ43 differs from Aβ42 by the presence of a threonine residue at the C-terminus. Overall, amyloid beta peptide means the peptide form that is involved in plaque formation that causes Alzheimer disease.

The term “half-life” is defined by the time taken for the removal of half the initial concentration of the fusion protein from the plasma. This invention describes ways of modulating the half-life in plasma. Such modification can produce fusion proteins with improved pharmacokinetic properties (e.g., increased in vivo serum half-life). Prolong the half-life means that it takes longer time to remove or get a clearance of half of the initial concentration of the fusion protein from the plasma. Half-life of a pharmaceutical or chemical compound is well defined and known in the art.

The term “connect” means a covalent or a reversible linkage between two or more parts. A covalent linkage can for example be a peptide bond, disulfide bond, carbon-carbon coupling or any type of linkage that is based of a covalent linkage between to atoms. Reversible linkage can for example be biotin-streptavidin, antibody-antigen or a linkage, which is classified as a reversible linkage known in the art. For example, a covalent linkage is directly obtained when the protein part and the modulator part of the fusion protein is produced in a recombinant form from the same plasmid, thus the connection is designed on DNA level.

The term “covalently connected” means a chemical link between two atoms in which electrons are shared between them. Examples of bonds covalently connected are a peptide bond, disulfide bond, carbon-carbon coupling. A fusion protein can be linked together by a polypeptide bond where the linkage can be accomplished during the translational process on the ribosome when the fusion protein are produced. Other type of covalently connected component could be modification with a pegylation reagent that is covalently linked to an amino residue (for example lysine) on the protein. The chemical coupling reaction can, for example, be acylation or other suitable coupling reaction which link the two components together into a fusion protein. Covalently connected can also mean a linkage of a linker at two sites in which the modulator is linked together with the protein part.

The term “cleavage sites” means a specific location/site in a peptide sequence that can be cleaved by a protein or an enzyme. Cleavage is normally produced by hydrolysis of the peptide bond connecting two amino acids. Cleavage can also take place at multiple sites in the same peptide using a single or a combination of proteins or enzymes. A cleavage site can also be other site than the peptide bond. This invention describes the cleavage of the amyloid β peptide in detail.

The term “binding domain” means a molecule that binds the amyloid β peptide with an affinity of that is therapeutically relevant. These molecules bind to amyloid β peptide with a binding affinity greater than or equal to about 10⁶, 10⁷, 10⁸, 10⁹, or 10¹⁰ M⁻¹. Typical binding domains are, but not restricted to, antibodies (e.g. Fab, scFv, single domains all including the CDR regions), scaffold proteins as described in this invention and in the literature or synthetically produced molecules with affinity for the amyloid β peptide.

The term “protease” means any protein molecule acting in the hydrolysis of peptide bonds. It includes naturally occurring proteolytic enzymes, as well as variants thereof obtained by site-directed or random mutagenesis or any other protein engineering method, any fragment of an proteolytic enzyme, or any molecular complex or fusion protein comprising one of the aforementioned proteins. The protease can be a serine, cysteine, aspartic or a metalloprotease.

The term “substrate” or “peptide substrate” means any peptide, oligopeptide, or protein molecule of any amino acid composition, sequence or length, that contains a peptide bond that can be hydrolyzed catalytically by a protease. The peptide bond that is hydrolyzed is referred to as the “cleavage site”. Numbering of positions in the substrate is done according to the system Introduced by Schlechter & Berger (Biochem. Biophys. Res. Commun. 27 (1967) 157-162). Amino acid residues adjacent N-terminal to the cleavage site are numbered P1, P2, P3, etc., whereas residues adjacent C-terminal to the cleavage site are numbered P1′, P2′, P3′, etc. The substrate or peptide substrate of this invention is the amyloid β peptide.

The term “specificity” means the ability of a protein or a protease to recognize and hydrolyze selectively certain peptide substrates while others remain uncleaved. Specificity can be expressed qualitatively and quantitatively. “Qualitative specificity” refers to the kind of amino acid residues that are accepted by a protease at certain positions of the peptide substrate. Proteases that accept only a small portion of all possible peptide substrates have a “high specificity”. Proteases that accept almost any peptide substrate have a “low specificity”. Proteases with very low specificity are also referred to as “unspecific proteases”.

The term “evolved protease” describes any protease that have been obtained using random PCR, DNA shuffling or other type of methods that generate diversity on the DNA/RNA level. Literature describing these approaches is for example; D. A. Drummond, B. L. Iverson, G. Georgiou and F. H. Arnold, Journal of Molecular Biology 350: 806-816 (2005) and S. McQ and D. S. Tawfik, Biochemistry 44: 5444-5452 (2005). Various approached to conduct screening and selection among the diversity created are also described in the literature (e.g. Directed Enzyme Evolution: Screening and Selection Methods (Methods in Molecular Biology) Editors: Frances H Arnold and George Georgiou. Volume 230, 2003 and references therein). Various strategies can be used to select for properties like increased stability, increased activity, improved selectivity and decreased inhibition by known and unknown inhibitors.

The term “improved protease” describes any protease variants that possess higher catalytic activity if that is needed. However, in some instances a lower catalytic activity might be preferable. Improved protease might also mean a variant that cleaves a certain substrate compared to another substrate more efficient that the original protease. Improved means a more preferred property, such as catalytic activity and/or selectivity to obtain a more optimized pharmaceutical compound. Improved protease can also mean variants with increased stability in for example plasma blood (both or either in vitro and in vivo). Improved protease can also mean variants with decreased inactivation in for example plasma blood (both or either in vitro or in vivo). Decreased inactivation can be accomplished by decreasing the protolytic degradation of the protease due to changed amino acid sequence, less prone to be cleaved. Decreased proteolytic degradation can also be accomplished by modifying the protein surface with for example pegylation and/or glycosylation to protect the protein from becoming cleaved. Decreased inactivation can also be accomplished by reducing inhibition of the protease by a known or unknown inhibitor. Reduced inhibition of an unknown blood plasma inhibitor can be accomplished by screening variants for reduced inhibition of protease activity directly in the blood plasma.

The term “human Neprilysin” refers to any natural form of human neprilysin. This includes all splice and polymorphic variants that naturally occur in the human population. A number of forms of human neprilysin are described in this invention (SEQ ID Nos 1 to 4). The term also include fragments or extended variants of human Neprilysin, as well as improved variants of human Neprilysin, as described under “improved protease”.

The term “scaffold protein” describes any protein that binds amyloid β peptide. Examples of scaffold proteins are tendamistat, affibody, anticalin and ankyrin. These scaffold proteins are typically designed and is based on a rigid core structure and a part, loops, surfaces or cavities that can be randomized for the identification of binders. These scaffold proteins are well described in the literature.

This invention suggests the possibility that the administration of an optimized recombinant Aβ degradation enzyme inhibits amyloid plaque formation by decreasing brain levels of Aβ. As a consequence, amyloid plaque-related astrogliosis will also be reduced.

In one aspect of this invention the therapeutic compound is of fully human origin. The fusion protein is composed of fully human proteins that are linked together using a linker with lowest possible immunogenic activity.

Advantages using a degrading enzyme compared to a binding molecule such an antibody are:
Degradation with an enzyme of the amyloid β peptide will directly remove the toxic effect compare to a binding approach where the concentration of the amyloid β peptide could potentially increase if the binding molecule in complex with amyloid β peptide is not cleared fast enough. This could be harmful especially if the amyloid β peptide concentration increases peripherally.
Catalytic degradation of amyloid β peptide will remove the peptide more efficiently that binding. Only a catalytic amount of the degrading enzyme will be necessary to remove sufficient amyloid β peptide whereas a binding molecule such as an antibody, a stoichiometric amount will be needed for a therapeutic effect. This will have a great impact on the amount needed for therapeutic treatment.
If the binding molecule is an antibody and cross the BBB allowing binding to the amyloid β peptide in the plaques, a potential immunological response that are harmful is possible. On the other hand, a catalytic fusion protein will not bind to the plaques and use the Fc reactivity but only reduce the free concentration of amyloid β peptide. Thus, A catalytic enzyme will only degrade the free pool of amyloid β peptide. A binding agent like an antibody could potentially enter the CNS and dissolve the plaques through Fc activity. This might be unfavorable if large amount of amyloid β peptide is released in the vicinity of the plaque and they are toxic to the cells.

One important enzyme in Aβ catabolism is Neprilysin, also known as neutral endopeptidase-24.11 or NEP. Iwata et al. (Nature Medicine, 6: 143-149, 2000) showed that the Aβ₁₋₄₂ peptide underwent full degradation through limited proteolysis conducted by NEP similar or identical to neprilysin as biochemically analysed. Consistently, NEP inhibitor infusion resulted in both biochemical and pathological deposition of endogeneous Aβ₄₂ in brain. It was found that this NEP-catalysed proteolysis therefore limits the rate of Aβ42 catabolism.

NEP is a 94 kD, type two membrane-bound Zn-metallopeptidase implicated in the inactivation of several biologically active peptides including enkephalins, tachykinins, bradykinin, endothelins and atrial natriuretic peptide. NEP is present in peptidergic neurons in the CNS, and its expression in brain is regulated in a cell-specific manner (Roques B. P. et al., Pharmacol. Rev. 45, 87-146, 1993; Lu B. et al., J. Exp. Med. 181, 2271-2275, 1995; Lu B. et al., Ann. N.Y. Acad. Sci. 780, 156-163, 1996). While type 2 NEP-transcripts are absent from the CNS, type 1 and type 3 transcripts are localized in neurons and in oligodendrocytes of the corpus callosum, respectively (Li C. et al., J. Biol. Chem. 270, 5723-5728, 1995). The Neprilysin family of proteases and endopeptidases comprises structurally or functionally homologous members of NEP such as the recently described NEP II gene and its isoforms (Ouimet T. et al., Biochem. Biophys. Res. Commun. 271:565-570, 2000), which are expressed in the CNS in a complementary pattern to NEP. A further member of this family is NL-1 (neprilysin like 1), a soluble protein efficiently inhibited by the NEP inhibitor phosphoramidon (Ghaddar G. et al., Biochem. J. 347: 419-429, 2000).

Other enzymes that are known to catabolise Aβ have also been described. The zinc metallopeptidase insulin-degrading enzyme (IDE, EC. 3.4.22.11) cleaves Aβ₁₋₄₀ and Aβ₁₋₄₂ into what appears to be innocuous products. IDE is a true peptidase; it does not hydrolyze proteins. The enzyme cleaves a limited number of peptides in vitro including insulin and insulin related peptides, β endorphin, and Aβ peptides. IDE has been suggested to be one of the physiological Aβ metabolizing enzymes (W. Q. Qui et al. (1998) J. Biol. Chem. 273, 32730-32738). Kurichkin and Goto (I. V. Kurochkin and S. Gato (1994) FEBS Lett. 345, 33-37) first reported that insulin degrading enzyme can hydrolyze Aβ₁₋₄₀. This finding was confirmed in two separate studies (W. Q. Qui et al. (1998) J. Biol. Chem. 273, 32730-32738; and J. R. McDermott and A. M. Gibson (1997) Neurochem. Res. 22, 49-56). Moreover, metalloprotease 24.15, a recently identified as a Aβ-degrading enzyme (Yamin R. et al., J. Biol. Chem. 274, 18777-18784, 1999), was also unchanged in response to Aβ injections. Angiotensin converting enzyme (ACE), an unrelated neuronal Zn-metalloendo peptidase have been also mention as a possible Aβ-peptide degrading enzyme (Barnes N. M. et al., Eur. J. Pharmacol. 200, 289-292, 1991; Alvarez R. et al., J. Neurol. Neurosurg. Psychiatry 67, 733-736, 1999; Amouyel P. et al., Ann. N.Y. Acad. Sci. 903, 437-441, 2000) with no known affinity to Aβ (McDermott J. R. and Gibson A. M., Neurochem. Res. 22, 49-56, 1997). Cathepsin B (CatB) have also been shown to degrade Aβ peptides (Neuron. 2006 Sep. 21; 51 (6):703-14).

The sequence used from the neprilysin may be the extracellular part of the protein. The extracellular part is defined as the part of neprilysin that is defined as outside the membrane region. This invention also includes the use of the whole sequence of neprilysin as the amyloid β peptide-degrading component. The invention also comprises smaller fragments of neprilysin as long as the catalytic activity is preserved against the amyloid β peptide. The invention also comprises any polymorphism variants and splice variants of neprilysin. The invention also comprises any improved variants of neprilysin.

This invention describes a novel and alternative strategy to hydrolyze Aβ peptides before they form amyloid plaques or at least prevent the further development of existing plaques. It may also be possible to remove existing plaques by hydrolyzing any plaque-derived Aβ peptide in equilibrium with free Aβ peptide.

Another embodiment of the present invention refers to a molecule that is composed of one part that binds amyloid β peptide with high affinity. This affinity is below micromolar in binding affinity. The binding affinity for amyloid β peptide is preferably at nanomolar in binding affinity. The other part that is involved in the interaction with amyloid β peptide is an active component that cleaves the amyloid β peptide at one or more site in the structure of the amyloid β peptide. The reason to combine a binding part linked together with a catalytic active part that both recognize the amyloid β peptide is that the binding part binds the amyloid β peptide and thereby increase the local concentration (the binding part and the catalytic part) is binding to the dissociated form of amyloid β peptide. Some bind specifically to the dissociated form without binding to the aggregated form. Some bind to both aggregated and dissociated forms. Some such antibodies bind to a naturally occurring short form of Aβ (i.e. covalently or in another way linked together) of amyloid β peptide to become cleaved by the active part that is locally around due to the linkage engineered in the bifunctional molecule. The linkage between the amyloid β peptide binding component and the amyloid β peptide-degrading component is preferably mediated by the plasma half-life modulator component with or without a linker component.

In some embodiments of this invention the therapeutic agents include fusion proteins that specifically bind to amyloid β peptide or other component of amyloid plaques. Such compound can be a part of a monoclonal or polyclonal or any other amyloid β peptide binding agent. These compounds bind to amyloid β peptide with a binding affinity greater than or equal to about 10⁶, 10⁷, 10⁸, 10⁹, or 10¹⁰ M⁻¹. These binding components are preferably connected with an amyloid β peptide-degrading component.

One aspect of the invention refers to the combination with the “Fc” domain of an antibody with a amyloid β peptide degrading component in the fusion protein. Antibodies comprise two functionally independent parts, a variable domain known as “Fab”, which binds antigen, and a constant domain known as “Fc”, which links to such effector functions as complement activation and attack by phagocytic cells. An Fc has a long serum half-life, whereas a Fab is short-lived (Capon et al. (1989), Nature 337: 525-31). When constructed together with a therapeutic protein, an Fc domain can provide longer half-life or incorporate such functions as Fc receptor binding, protein A binding, complement fixation and perhaps even placental transfer.

Preferred molecules in accordance with this invention are Fc-linked amyloid β peptide degrading protein such as NEP-related proteins.

Useful modifications of protein therapeutic agents by fusion with the Fc domain of an antibody are discussed in detail in a publication entitled, “Modified Peptides as Therapeutic Agents (WO 99/25044). That publication discusses linkage to a “vehicle” such as PEG, dextran, or an Fc region. Linking to the C-terminal part of an Fc domain has been described in the literature as a possible approach (Protein Eng. 1998 11:495-500). This allows a N-terminal linkage on the protein part of the fusion protein. This invention describes this approach and the beneficial effect of using this strategy obtaining a fusion protein with optimized properties for in vivo efficacy.

IgG molecules interact with three classes of Fc receptors (FcR) specific for the IgG class of antibody, namely FcγRI, FcγRII and FcγRIII. In preferred embodiments, the immunoglobulin (Ig) component of the fusion protein has at least a portion of the constant region of an IgG that has a low binding affinity for at least one of FcγRI, FcγRII or FcγRIII. In one aspect of the invention, the binding affinity of fusion proteins for Fc receptors is reduced by using heavy chain isotypes as fusion partners that have reduced binding affinity for Fc receptors on cells. For example, both human IgG1 and IgG3 have been reported to bind to FcRγI with high affinity, while IgG4 binds 10-fold less well, and IgG2 does not bind at all. The important sequences for the binding of IgG to the Fc receptors have been reported to be located in the CH2 domain. Thus, in a preferred embodiment, an antibody-based fusion protein with enhanced in vivo circulating half-life is obtained by linking at least the CH2 domain of IgG2 or IgG4 to a second non-immunoglobulin protein. For example, of the four known IgG isotypes, IgG1 (Cγ1) and IgG3 (Cγ3) are known to bind FcRγI with high affinity, whereas IgG4 (Cγ4) has a 10-fold lower binding affinity, and IgG2 (Cγ2) does not bind to FcRγI.

In one embodiment, the Aβ-peptide degrading component of the fusion protein is an enzyme. The term “enzyme” is used herein to describe proteins, analogs thereof, and fragments thereof, which are active as proteases or petidases. Preferably, enzymes include serine, aspartic, metallo and cysteine proteases. Preferably, the fusion protein of the present invention displays enzymatic biological activity.

In another embodiment, the immunoglobulin domain is selected from the group consisting of the Fc domain of IgG, the heavy chain of IgG, and the light chain of IgG. In another embodiment, the constant region of the antibody in the fusion protein will be of human origin, and belong to the immunoglobulin family derived from the IgG class of immunoglobulins, in particular from classes IgG1, IgG2, IgG3 or IgG4, preferably from the class IgG2 or IgG4. It is also alternatively possible to use constant regions of immunoglobulins belonging to the IgG class from other mammals, in particular from rodents or primates; however, it is also possible, according to the invention, to use constant regions of the immunoglobulin classes IgD, IgM, IgA or IgE. Typically, the antibody fragments that are present in the construct according to the invention will comprise the Fc domain CH₃, or parts thereof, and at least one part segment of the Fc domain CH₂. Alternatively, it is also possible to conceive of fusion constructs according to the invention which contain, as component (A), the CH₃ domain and the hinge region, for the dimerization.

However, it is also possible to use derivatives of the immunoglobulin sequences that are found in the native state, in particular those variants that contain at least one replacement, deletion and/or insertion (combined here under the term “variant”). Typically, such variants possess at least 90%, preferably at least 95%, and more preferably at least 98%, sequence identity with the native sequence. Variants, which are particularly preferred in this context, are replacement variants that typically contain less than 10, preferably less than 5, and very particularly preferably less than 3, replacements as compared with the respective native sequence. Attention is drawn to the following replacement possibilities as being preferred: Trp with Met, Val, Leu, Ile, Phe, His or Tyr, or vice versa; Ala with Ser, Thr, Gly, Val, Ile or Leu, or vice versa; Glu with Gln, Asp or Asn, or vice versa; Asp with Glu, Gln or Asn, or vice versa; Arg with Lys, or vice versa; Ser with Thr, Ala, Val or Cys, or vice versa; Tyr with His, Phe or Trp, or vice versa; Gly or Pro with one of the other 19 native amino acids, or vice versa.

Soluble receptor-IgG fusion proteins are common immunological reagents and methods for their construction are known in the art (see e.g., U.S. Pat. No. 5,225,538). A functional amyloid β peptide-degrading domain may be fused to an immunoglobulin Fc domain derived from an immunoglobulin class or subclass. The Fc domains of antibodies belonging to different Ig classes or subclasses can activate diverse secondary effector functions. Activation occurs when the Fc domain is bound by a cognate Fc receptor. Secondary effector functions include the ability to activate the complement system, to cross the placenta, and to bind various microbial proteins. The properties of the different classes and subclasses of immunoglobulins are described in Roitt et al., Immunology, p. 4.8 (Mosby-Year Book Europe Ltd., 3d ed. 1993). The Fc domains of antigen-bound IgG1, IgG3 and IgM antibodies can activate the complement enzyme cascade. The Fc domain of IgG2 appears to be less effective, and the Fc domains of IgG4, IgA, IgD and IgE are ineffective at activating complement. Thus one can select an Fc domain based on whether its associated secondary effector functions are desirable for the particular immune response or disease being treated with the amyloid β peptide degrading-Fc fusion protein. If it would be advantageous to harm or kill target cells, one could select an especially active Fc domain (IgG1) to make the amyloid β peptide degrading-Fc-fusion protein. Alternatively, if it would be desirable to produce the amyloid β peptide degrading-Fc-Fusion without triggering the complement system, an inactive IgG4 Fc domain could be selected. This invention describes a fusion protein with a catalytic component linked to a Fc part and not a direct binding component. This means that the effect and activity from the Fc will be limited because many Fc effects are mediated through the binding. For example complement activation is dependent on binding and the formation of a network.

C-terminally of the immunoglobulin fragment, a fusion construct according to the invention typically, but not necessarily, contains a transition region between catalytic and modulator part, which transition region can in turn contain a linker sequence, with this linker sequence preferably being a peptide sequence. This peptide sequence can have a length from between 1 and up to 70 amino acids, where appropriate even more amino acids, preferably from 10 to 50 amino acids, and particularly preferably between 12 and 30 amino acids. The linker region of the transition sequence can be flanked by further short peptide sequences which can, for example, correspond to DNA restriction cleavage sites. Any restriction cleavage sites with which the skilled person is familiar from molecular biology can be used in this connection. Suitable linker sequences are preferably artificial sequences which contain a high number of proline residues (for example at every second position in the linker region) and, in addition to that, preferably have an overall hydrophilic character. A linker sequence, which consists of at least 30% of proline residues, is preferred. The hydrophilic character can preferably be achieved by means of at least one amino acid having a positive charge, for example lysine or arginine, or negative charge, for example aspartate or glutamate. Overall, the linker region therefore also preferably contains a high number of glycine and/or proline residues in order to confer on the linker region the requisite flexibility and/or rigidity.

However, native sequences, for example those fragments of ligands belonging to the NEP family which are disposed extracellularly, but immediately act, i.e. in front of, the cell membrane, are also suitable for use as linkers, where appropriate after replacement, deletion or insertion of the native segments as well. These fragments are preferably the 50 AA which follow extracellularly after the transmembrane region or else subfragments of these first 50 AA. However, preference is given to these segments having at least 85% sequence identity with the corresponding natural human sequences, with very particular preference being given to at least 95% sequence identity and particular preference being given to at least 99% sequence identity in order to limit the immunogenicity of these linker regions in the fusion protein according to the invention and not elicit any intrinsic humoral defense reaction. Within the context of the present invention, the linker region should preferably not possess any immunogenicity.

However, as an alternative to peptide sequences which are linked to the amyloid β peptide degrading component and the plasma half-life modulator component, by way of amide-like bonds, it is also possible to use compounds which are of a nonpeptide or pseudopeptide nature or are based on noncovalent bonds. Examples which may be mentioned in this connection are, in particular, N-hydroxysuccinimide esters and heterobifunctional linkers, such as N-succinimidyl-3-(2-pyridyldi-thio) propionate (SPDP) or similar crosslinkers.

Other ways of regulating the plasma half-life is to use pegylation or other type of modifications that increasing the molecular weight such as glycosylation.

As noted above, polymer modulators may also be used. Various means for attaching chemical moieties useful as modulator are currently available, see, e.g., patent application WO 96/11953, entitled “N-Terminally Chemically Modified Protein Compositions and Methods,” herein incorporated by reference in its entirety. This PCT publication discloses, among other things, the selective attachment of water-soluble polymers to the N-terminus of proteins.

A preferred polymer modulator is polyethylene glycol (PEG). The PEG group may be of any convenient molecular weight and may be linear or branched. The average molecular weight of the PEG will preferably range from about 2 kiloDalton (“kD”) to about 100 kDa, more preferably from about 5 kDa to about 50 kDa, most preferably from about 5 kDa to about 10 kDa. The PEG groups will generally be attached to the compounds of the invention via acylation or reductive alkylation through a reactive group on the PEG moiety (e.g., an aldehyde, amino, thiol, or ester group) to a reactive group on the compound (e.g. an aldehyde, amino, or ester group).

A useful strategy for the PEGylation of protein consists of combining, through forming a conjugate linkage in solution, a protein and a PEG moiety, each bearing a special functionality that is mutually reactive toward the other. The protein can be prepared with conventional recombinant expression techniques. The proteins are “preactivated” with an appropriate functional group at a specific site. The precursors are purified and fully characterized prior to reacting with the PEG moiety. Ligation of the protein with PEG usually takes place in aqueous phase and can be easily monitored by reverse phase analytical HPLC. The PEGylated protein can be easily purified by preparative HPLC and characterized by analytical HPLC, amino acid analysis and laser desorption mass spectrometry.

Polysaccharide polymers are another type of water-soluble polymer which may be used for protein modification. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water-soluble polymer for use in the present invention as a modulator by itself or in combination with another modulator (e.g., Fc), see e.g. WO 96/11953 and WO 96/05309. The use of dextran conjugated to therapeutic or diagnostic immunoglobulins has been reported; see, for example, European Patent Publication EP 0 315 456, which is hereby incorporated by reference. Dextran of about 1 kD to about 20 kD is preferred when dextran is used as a vehicle in accordance with the present invention.

Carbohydrate (oligosaccharide) groups may conveniently be attached to sites that are known to be glycosylation sites in proteins. Generally, O-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art. Amino acids that are suitable for glycosylation can be incorporated at specific sites both in the modulator and the protein part. Preferable techniques to use for engineering these specific amino acids are site-directed mutagenesis or comparable method. Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983). Thus, glycosylation sites in the amyloid β peptide degrading component can be engineered. For example, residues preferably on the surface of neprilysin structure are modified to allow the glycosylation. The 3D structure of neprilysin is know an can be used to select suitable amino acid replacement for the introduction of both glycosylation and pegylation sites. Glycosylation sites are introduced using for example the Asn-X-Ser/Thr sequence. For pegylation, suitable surface exposed amino acids are for example replaced to cystine residues for specific and efficient coupling of the pegylation component.

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Linkers: Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines.

The quantitative specificity of proteases varies over a wide range. There are very unspecific proteases known, such as papain which cleaves all polypeptides that contain a phenylalanine, a valine or an leucine residue, or trypsin which cleaves all polypeptides that contain an arginine or a lysine residue. On the other hand, there are highly specific proteases known, such as the tissue-type plasminogen activator (t-PA) which cleaves plasminogen only at a single specific sequence. Proteases with high substrate specificity play an important role in the regulation of protein functions in living organisms. The specific cleavage of polypeptide substrates, for example, activates precursor proteins or deactivates active proteins or enzymes, thereby regulating their functions. Several proteases with high substrate specificities are used in medical applications. Pharmaceutical examples for activation or deactivation by cleavage of specific polypeptide substrates are the application of t-PA in acute cardiac infarction, which activates plasminogen to resolve fibrin clots, or the application of Ancrod in stroke which deactivates fibrinogen, thereby decreasing blood viscosity and enhancing its transport capacity. While t-PA is a human protease with an activity necessary in human blood regulation, Ancrod is a non-human protease. It was isolated from the viper Agkistrodon rhodostoma, and comprises the main ingredient of the snake's poison. Therefore, there exist a few non-human proteases with therapeutic applicability. Their identification, however, is usually highly incidental. The treatment of diseases by administering drugs is typically based on a molecular mechanism initiated by the drug that activates or inactivates a specific protein function in the patient's body, be it an endogenous protein or a protein of an infecting microbe or virus. While the action of chemical drugs on these targets is still difficult to understand or to predict, protein drugs are able to specifically recognize these target proteins among millions of other proteins. Prominent examples of proteins that have the intrinsic possibility to recognize other proteins are antibodies, receptors, and proteases. Although there are a huge number of potential target proteins, only very few proteases are available today to address these target proteins. Due to their proteolytic activity, proteases are particularly suited for the inactivation of protein or peptide targets. When considering human proteins only, the number of potential target proteins is yet enormous. It is estimated that the human genome comprises between 30,000 and 100,000 genes, each of which encodes a different protein. Many of these proteins or peptides are involved in human diseases and are therefore potential pharmaceutical targets. It might be unlikely to find such a protease with a particular qualitative specificity by screening natural isolates. Therefore there is a need to optimize the catalytic selectivity of a known protease or other scaffold proteins including catalytic antibodies.

Selection systems for proteases of known specificity are known in the art, for instance, from Smith et al., Proc. Natl. Acad. Sci USA, Vol. 88 (1991). As exemplified, the system comprises the yeast transcription factor GAL4 as the selectable marker, a defined and cleavable target sequence inserted into GAL4 in conjunction with the TEV protease. The cleavage separates the DNA binding domain from the transcription activation domain and therewith renders the transcription factor inactive. The phenotypical inability of the resulting cells to metabolize galactose can be detected by a calorimetric assay or by the selection on the suicide substrate 2-deoxygalactose.

Further, selection may be performed by the use of peptide substrates with modifications as, for example, fluorogenic moieties based on groups as ACC, previously described by Harris et al. (US 2002/022243).

Identical or similar approaches could be used in order to identify or produce an effective amyloid β peptide-degrading component as described in this invention. That starting point for the engineering of this amyloid β peptide-degrading component could be an enzyme that possesses some activity against amyloid β peptide or that have no activity at all. Other components could be a scaffold protein where specific regions are randomized to possess activity against the amyloid β peptide. There are described various scaffold proteins in the literature where one part of the scaffold structure is the core structure holding the randomized part in a relative fixed positions to generate a binding or active site. Enzymes that possess some activity against amyloid β peptide could be natural proteases that are described to degrade amyloid β peptide. For example, neprilysin could be engineered either by rationale design or a more random approach to become more efficient as a amyloid β peptide-degrading component.

Laboratory techniques to generate proteolytic enzymes with altered sequence specificities are in principle known. They can be classified by their expression and selection systems. Genetic selection means to produce a protease or any other protein within an organism which protease or any other protein is able to cleave a precursor protein which in turn results in an alteration of the growth behavior of the producing organism. From a population of organisms with different proteases those having an altered growth behavior can be selected. This principle was reported by Davis et al. (U.S. Pat. No. 5,258,289). The production of a phage system is dependent on the cleavage of a phage protein, which is activated in the presence of a proteolytic enzyme, or antibody which is able to cleave the phage protein. Selected proteolytic enzymes, scaffolds or antibodies would have the ability to cleave an amino acid sequence for activation of phage production.

A system to generate proteolytic enzymes with altered sequence specificities with membrane-bound proteases is reported. Iverson et al. (WO 98/49286) describe an expression system for a membrane-bound protease which is displayed on the surface of cells. An essential element of the experimental design is that the catalytic reaction has to be performed at the cell surface, i.e., the substrates and products must remain associated with the bacterium expressing the enzyme at the surface. Another example of a selection system is the use of FACS sorting (Varadarajan et al., Proc. Natl. Acad. Sci. USA, Vol. 102, 6855 (2005)) that express the active protein on a cell surface and sort cells that contains variants with improved properties. They showed a three million-fold change in specificity for a protease cleavage site.

A system to generate proteolytic enzymes with altered sequence specificities with self-secreting proteases is also known. Duff et al. (WO 98/11237) describe an expression system for a self-secreting protease. An essential element of the experimental design is that the catalytic reaction acts on the protease itself by an autoproteolytic processing of the membrane-bound precursor molecule to release the matured protease from the cellular membrane into the extracellular environment.

Broad et al. (WO 99/11801) disclose a heterologous cell system suitable for the alteration of the specificity of proteases. The system comprises a transcription factor precursor wherein the transcription factor is linked to a membrane-anchoring domain via a protease cleavage site. The cleavage at the protease cleavage site by a protease releases the transcription factor, which in turn initiates the expression of a target gene being under the control of the respective promotor. The experimental design of alteration of the specificity consists in the insertion of protease cleavage sites with modified sequences and the subjection of the protease to mutagenesis.

According to the invention, any protein or peptide can be used directly or as a starting point to generate a suitable amyloid β peptide-degrading component. For example, according to the invention, any protease can be used as first protease. Preferably, any protein or peptide that are of human origin is used. If a natural protein or peptide, normally existing in the human body, is used, the smallest possible changes are preferred. In some methods, two or more fusion proteins with different binding specificities and/or degradation activity are administered simultaneously, in which case the dosage of each fusion protein administered falls within the ranges indicated. Fusion protein is usually administered on multiple occasions. Intervals between single dosages can be, for example, weekly, monthly, every three months or yearly. Intervals can also be irregular as indicated by measuring blood levels of fusion protein in the plasma of the patient. In some methods, dosage is adjusted to achieve a plasma fusion protein concentration of 1-1000 ug/ml and in some methods 25-300 ug/ml. Also in some methods, dosage is adjusted to achieve a plasma fusion protein concentration of 1-1000 ng/ml and in some methods 25-300 ng/ml. Alternatively, fusion protein can be administered as a sustained release formulation, in which case less frequent administration is required. Dosage and frequency vary depending on the half-life of the fusion protein in the patient. In general, fusion protein with an Fc part shows a long half-life. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime. It is predicted that a catalytic active amyloid β peptide degrading fusion protein can be administrated at a lower dose compare to a binding agent such as for example an antibody.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

One aspect of the present invention is the possibility to modify natural wild type proteins to become even more selective in the degradation of amyloid β peptide. Site-directed mutagenesis can be used to introduce/replace amino acids in the wild type sequence. On approach is to use rational design by investigating the active site of the degrading enzymes. Amino acids that potentially will alter the selectivity profile (degradation of amyloid β peptide compare to other peptides/proteins) can be replace with other amino acids a the new variants can be tested in cleavage assays known in the art. Preferably, variants that have a higher catalytic degradation activity towards amyloid β peptide compare to other related peptides are useful. Other related peptides include but are not limited to Enkephalin, Neuropeptide Y, Substance P, somastatin and cholecystokinin.

The three-dimensional structure of neprilysin is known (Oefner et al (2000) J. Mol. Biol. 296:341-9; Sahli et al. (2005) Helv. Chim. Acta. 88:731). This structure can guide the way changes are introduced in the structure and also which part that are most efficient to change in order to make libraries for screening or selection. The active site of neprilysin is very deeply buried in the structure explaining the enzymes preference for small substrate such a peptide fragments with a molecular weight below about 5000 Da. The active site residues include N542, H583, H587, E646 and R717. Amino acid residues close to the active site also include V580, F563, F564, M579, F716, I718, F106, I558, F563, F579, V580, H583, V692, W693 and A543 (Voisin et al (2004) JBC 279:46172-81). These and other residues can be changed by rationale design investigating the three-dimensional structure, or be randomly changed in a various libraries to obtained improved variants of neprilysin.

It is an object of the present invention to provide methods and materials, which are suited for the development of a treatment for neurodegenerative diseases and for the identification of compounds useful for therapeutic intervention in such diseases. Based on the finding that β-amyloid can be clearance through an optimized enzymatic-mediated mechanism the present invention sets out for providing such methods and materials as laid out in the claims section and described hereinafter.

The invention provides a method for preventing and treating neurodegenerative disorders comprising administering to the peripheral system of a mammalian an effective amount of an optimized enzymatic active compound. In particular, the enzymatic active compound is a fusion protein where one part has enzymatic activity and the other part regulate the half-life in plasma. The method is suited for preventing and treating brain amyloidosis such as Alzheimer's disease. The invention also provides different assay principles—biochemical and in particular cellular assays for testing an optimized enzymatic compound, preferably screening a plurality of compounds, for modulating activity and plasma half-life. In a further embodiment, the assay comprises the addition of a known inhibitor of the member of the neprilysin family before detecting said enzymatic activity. Suitable inhibitors are e.g. phosphoramidon, thiorphan, spinorphin, or a functional derivative of the foregoing substances.

In a general sense, assays according to the invention measure the enzymatic activity and half-life in plasma, both in vitro and in vivo.

In another aspect, the present invention provides a method for producing a medicament comprising the steps of (i) identifying a compound which degrades Aβ-peptides, preferably a compound that is highly specific and with high Aβ-peptides degrading activity (ii) linking this Aβ-peptides degrading compound to a modulator compound that determine the half-time in plasma.

The compounds of this invention may be made in transformed host cells using recombinant DNA techniques. To do so, a recombinant DNA molecule coding for the fusion protein is prepared. Methods of preparing such DNA molecules are well known in the art. For instance, sequences coding for the modulator and protein could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the modulator, protein or fusion in an appropriate host. The vector comprises the DNA molecule that codes for the modulator, protein and/or fusion operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art. Any of a large number of available and well-known host cells may be used in the practice of this invention. The selection of a particular host is dependent upon a number of factors recognized by the art. These include, for example, compatibility with the chosen expression vector, toxicity of the fusion encoded by the DNA molecule, rate of transformation, ease of recovery of the fusion, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the fusion is purified from culture by methods well known in the art. One preferably approach is to use Protein A or similar technique to purify the fusion protein when using a Fc part as a modulator. The modulator, protein and fusion may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides or proteins since it is the most cost-effective method of making small peptides or proteins.

In general, the compounds of this invention have pharmacologic activity resulting from their ability to degrade the amyloid β peptide in vivo. The activity of these compounds can be measured by assays known in the art. For the Fc-NEP compounds, in vivo assays are further described in the Examples section herein.

In general, the present invention also provides the possibility of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g. Remington's Pharmaceutical Sciences, 18th Ed. (1990, Mack Publishing Co., Easton, Pa. 18042) pages 1435-1712 which are herein incorporated by reference. The compositions may be prepared in liquid form, or may be in dried powder, such as lyophilized form. Implantable sustained release formulations are also contemplated, as are transdermal formulations. These administration alternatives are well known in the art.

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

This invention describe clearly that an amyloid β degrading protein can be modified in a specific way to maintain significant degrading activity and become suitable for in vivo usage. Experimental evidence is disclosed supporting this invention.

In some embodiments, the present invention provides a method for the treatment of Aβ-related pathologies such as Downs syndrome and β-amyloid angiopathy, such as but not limited to cerebral amyloid angiopathy, systemic amyloidosis, hereditary cerebral hemorrhage, disorders associated with cognitive impairment, such as but not limited to MCI (“mild cognitive impairment”), Alzheimer Disease, memory loss, attention deficit symptoms associated with Alzheimer disease, neurodegeneration associated with diseases such as Alzheimer disease or dementia including dementia of mixed vascular and degenerative origin, pre-senile dementia, senile dementia and dementia associated with Parkinson's disease, progressive supranuclear palsy or cortical basal degeneration, comprising administering to a mammal (including human) a therapeutically effective amount of a fusion protein according to the present invention.

### EXAMPLES

The invention is herein described by the following, non-limiting examples:

### Example 1

### Description of the Protein Domains

The extracellular domain of Neprilysin is defined as amino acid 51-749 (excluding the first Methionine) (SEQ ID NOS 1-4). There are two polymorphisms that lead to amino acid difference identified in this domain, and the amino acid sequence for the different variants are described in SEQ ID NO 1-4.

IDE (insulin degrading enzyme) is a 1018 amino acid long protein (SEQ ID NO 5). There are splice variants and polymorphism variants described of IDE. In one splice variant, one exon is replaced with another exon of the same size, encoding a peptide sequence similar to the “wt” exon (described in SEQ ID NO 6). This variant has been described to be less efficient in degrading both insulin and Aβ. There are also several polymorphisms in the IDE gene described, that lead to amino acid difference identified in this domain: D947N, E612K, L298F and E408G (numbering according to SEQ ID NO 5). All combinations of these polymorphisms are also possible.

The extra-cellular domain of ECE1 (endothelin-converting enzyme 1) (SEQ ID NO 7) is a 681 amino acids long protein, defined as amino acid 90-770 of the full-length, membrane-bound ECE1 protein. The ECE1 gene contains several possible polymorphisms that lead to amino acid difference: R665C, W541R, L494Q and T252I. All combinations of these polymorphisms are also possible.

The extracellular domain of Neprilysin, IDE and ECE1 are fused to the human IgG2 Fc domain (including the hinge region). A signal sequence (SEQ ID NO 8) is introduced to enable secretion of the protein into the culture media during expression. The sequence of the hinge region is shown in SEQ ID NO 9 and the IgG2 Fc domain is shown in SEQ ID NO 10. The complete fusion proteins (excluding the signal sequence) with a human Neprilysin variant corresponding to SEQ ID NO 1, IDE corresponding to SEQ ID NO 5 and ECE1 corresponding to SEQ ID NO 7, are described in SEQ ID NOS 11-13. The final fusion proteins (excluding the signal sequence) have predicted molecular weights of 211 kDa (Fc-Nep as a dimer), 294 kDa (Fc-IDE as a dimer) and 206 kDa (Fc-ECE1 as a dimer).

### Example 2

### Description of the Construction of the Gene Encoding the Fusion Protein Fc-Neprilysin

The gene encoding the extra-cellular domain of Neprilysin as fusion to the gene encoding Fc domain of IgG2, was synthetically made (GeneArt). The complete gene (encoding the Fc-Neprilysin) including the signal sequence was transferred from the GeneArt vector (pCR-Script, pGA4 or pUC-Kana) to a Gateway donor vector. The Gateway donor vectors are used to introduce the complete gene into several expression vectors. By using the Gateway system, the transfer from donor vectors to the expression vectors could be done by using recombination instead of restriction enzymes. The mammalian expression vectors investigated were primarily pCEP4, pEAK10, pEF5/FRT/V5-DEST and pcDNA5/FRT/TO (Gateway adapted). All these are standard mammalian expression vectors based on a CMV promoter (pCEP4, pEAK10 and pcDNA5/FRT/TO) or EF-1α promotor (pEF5/FRT/V5-DEST). The genes were sequenced after all cloning steps to verify the DNA sequence.

### Example 3

### Description of the Construction of the Genes Encoding the Fusion Proteins Fc-IDE and Fc-ECE1

The gene encoding the enzymes IDE and ECE1 as fusions to the gene encoding Fc domain of IgG2, are synthetically made. The complete genes (encoding the Fc-IDE and Fc-ECE1 fusion protein including the signal sequence) are transferred from the initial cloning vectors (pCR-Script, pGA4 or pUC-Kana) to a Gateway donor vector. The Gateway donor vectors are used to introduce the complete gene into several expression vectors. The mammalian expression vectors investigated are primarily pCEP4, pEAK10, pEF5/FRT/V5-DEST and pcDNA5/FRT/TO (Gateway adapted). All these are standard mammalian expression vectors based on a CMV promoter (pCEP4, pEAK10 and pcDNA5/FRT/TO) or EF-1α promotor (pEF5/FRT/V5-DEST). The genes are sequenced after all cloning steps to verify the DNA sequence.

### Example 4

### Expression of Extra-Cellular Domain of Neprilysin and Fusion Protein Fc-Neprilysin in HEK293 Cells

The protein Neprilysin (extra-cellular domain only) and Fc-Neprilysin (Fc-Nep) were transiently expressed in suspension-adapted mammalian cells. The cell lines used in the production experiments were cell lines derived from HEK293, including HEK293S, HEK293S-T and HEK293S-EBNA cells. Expression from plasmids pCEP4 and pEAK10 encoding the protein of interest was tested. Transfection was performed at cell density of approximately 0.5-×10⁶ and with plasmid DNA at concentrations ranging from 0.3-0.8 μg/ml cell suspension (final concentration). Tested transfection reagents are Polyethylenimine (Polyscience) at 2 μg/ml cell suspension (final concentration). Expression was performed in cell culture volumes of 30 ml to 1000 ml (shaker flasks), and 5 L to 10 L Wave Bioreactor. Expression was followed by taking samples from the culture supernatants at different days and analyzing cell density, cell viability, protein expression and enzyme activity. Cell cultures were harvested after 4 to 14 days by centrifugation. The cell culture media was used in protein purification experiments. All plasmid concentrations and vectors were successful, giving different levels of production, typically in the range of 1-3 mg/L.

### Example 5

### Expression of Fusion Proteins Fc-IDE and FcECE1 in HEK293 Cells

The proteins Fc-IDE and Fc-ECE1 are transiently expressed in suspension-adapted mammalian cells. The cell lines used in the production experiments are cell lines derived from HEK293, including HEK293S, HEK293S-T and HEK293S-EBNA cells. Expression from plasmids pCEP4 and pEAK10 encoding the protein of interest is tested. Transfection is performed at cell density of approximately 0.5-1×10⁶ and with plasmid DNA at concentrations ranging from 0.3-0.8 μg/ml cell suspension (final concentration). Tested transfection reagents are Polyethylenimine (Polyscience) at 2 μg/ml cell suspension (final concentration). Expression is performed in cell culture volumes of 30 ml to 1000 ml (shaker flasks), and 5 L to 10 L in Wave Bioreactor. Expression is followed by taking samples from the culture supernatants at different days and analyzing cell density, cell viability, protein expression and enzyme activity. Cell cultures are harvested after 4 to 14 days by centrifugation. The cell culture media is used in protein purification experiments.

### Example 6

### Expression of Extra Cellular Domain of Neprilysin and Fusion Protein Fc-Neprilysin in CHO-S Cells

The proteins Neprilysin (extra cellular domain only) and Fc-Nep were stably expressed in suspension-adapted mammalian cells. The host cells used in the production experiments were the FlpIn CHO-cells (Invitrogen), which have been adapted to suspension growth. Expression from plasmids pcDNA5/FRT/TO-DEST30 and pEF5/FRT/V5-DEST encoding the protein of interest was tested. The expression was driven by either the CMV promoter or the EF1alpha promoter. Transfection was performed at a cell density of approximately 1×10⁶ cells/ml in F12 media using plasmid DNA at concentrations of about 0.1 μg/ml (final concentration). A helper plasmid pOG44 coding for a recombinase was cotransfected at a final concentration of 0.8 μg/ml. Polyethylenimine (Polyscience) at 2 μg/ml cell suspension (final concentration) was used as transfection reagent. Expression was performed in cell culture volumes of 30 ml to 1000 ml in shaker flasks. Samples from the culture supernatants were taken at different days and cell density, cell viability, protein expression and enzyme activity were analyzed. Cell cultures were harvested after 4 to 11 days by centrifugation. Finally, the cell culture media was used in protein purification experiments. Both expression vectors used were successful in producing the desired proteins. The production levels were typically in the range of 10-50 mg/L.

### Example 7

### Expression of Fusion Protein Fc-IDE and Fc-ECE1 in Cho-S Cells

The proteins Fc-IDE and Fc-ECE1 are stably expressed in suspension-adapted mammalian cells. The host cells used in the production experiments are the FlpIn CHO-cells (Invitrogen), which have been adapted to suspension growth. Expression from plasmids pcDNA5/FRT/TO-DEST30 and pEF5/FRT/V5-DEST encoding the protein of interest is tested. The expression is driven by either the CMV promoter or the EF1 alpha promoter. Transfection is performed at a cell density of approximately 1×10⁶ cells/ml in F12 media using plasmid DNA at concentrations of about 0.1 μg/ml (final concentration). A helper plasmid pOG44 coding for a recombinase is cotransfected at a final concentration of 0.8 μg/ml. Polyethylenimine (Polyscience) at 2 μg/ml cell suspension (final concentration) is used as transfection reagent. Expression is performed in cell culture volumes of 30 ml to 1000 ml in shaker flasks. Samples from the culture supernatants are taken at different days and cell density, cell viability, protein expression and enzyme activity are analyzed. Cell cultures are harvested after 4 to 11 days by centrifugation.

### Example 8

### Purification of Expressed Fc-Neprilysin Protein by Affinity Chromatography

Purification of the fusion protein was performed using cell media from expression in mammalian cells. The purification was performed by Affinity chromatography (Protein A) followed by low pH elution, and was performed on ÄKTA Chromatography systems (Explorer or Purifier, GE Healthcare). rProtein A Sepharose FF (GE Healthcare) in an XK26 column (GE Healthcare) was equilibrated with 10 column volumes (CV) of PBS (2.7 mM KCl, 138 mM NaCl, 1.5 mM KH₂PO₄, 8 mM Na₂HPO₄-7H₂O, pH 6.7-7.0, Invitrogen). Cell culture media with expressed fusion protein (Fc-Neprilysin) was applied on the column. The column was washed with 20 CV PBS before bound protein was eluted with Elution buffer (0.1 M Glycine, pH 3.0). Purified fractions were immediately neutralized by adding 50 μl of 1M Tris Base to 1 ml of eluted protein. Purified fractions were pooled and buffer was exchanged to 50 mM Tris-HCl, pH 7.5, 150 mM NaCl using PD10 Columns (GE Healthcare). Purified protein was analyzed on SDS-PAGE, and was found to be approximately 90% pure.

### Example 9

### Purification of Expressed Fc-IDE and Fc-ECE1 by Affinity Chromatography

Purification of the fusion protein is performed using cell media from expression in mammalian cells. rProtein A Sepharose FF (GE Healthcare) in an XK26 column (GE Healthcare) is equilibrated with 10 column volumes (CV) of PBS (2.7 mM KCl, 138 mM NaCl, 1.5 mM KH₂PO₄, 8 mM Na₂HPO₄-7H₂O, pH 6.7-7.0, Invitrogen). Cell culture media with expressed fusion protein (Fc-IDE or Fc-ECE1) is applied on the column. The column is washed with 20 CV PBS before bound protein is eluted with Elution buffer (0.1 M Glycine, pH 3.0). Purified fractions are immediately neutralized by adding 50 μl of 1M Tris Base to 1 ml of eluted protein. Purified fractions are pooled and buffer is exchanged to 50 mM Tris-HCl, pH 7.5, 150 mM NaCl using PD10 Columns (GE Healthcare).

### Example 10

### SDS-PAGE and Western Blot Analysis of Expression of Fc-Neprilysin

Cell culture media from expression in mammalian cells was analyzed using western blot. 20 μl of cell culture media was diluted in 4×LDS Sample Buffer (Invitrogen) including Sample Reducing Agent (Invitrogen). The samples were heated to 95° C. for 5 minutes and loaded on an SDS-PAGE gel (4-12% Gradient gel, 10 wells (1 mm), Invitrogen). MES Buffer was used as running buffer. The gels were run at 200 V for 35 minutes. Electro blotting was performed at 30 V for 1 hour, to transfer the proteins to PVDF membranes. The membranes were blocked in TBST (TBS (20 mM Tris, 500 mM NaCl, pH 7.5 (BioRad) plus 0.05% Tween-20) including 5% BSA overnight, before they were incubated with 30 μl of primary antibody (Biotinylated Goat Anti-human Neprilysin Antibody, 50 μg/ml (R&D Systems)) in 15 ml TBST. The membranes were incubated in room temperature for two hours, washed three times with TBST, and incubated for one hour with Streptavidin-horseradish peroxidase conjugate (GE Healthcare, diluted 1:10 000 (1.5 μl in 15 ml TBST)). The membranes were washed three times with TBST and three times with water before the bands were visualized using ECL plus reagent (GE Healthcare) and ECL films (GE Healthcare). SDS-PAGE showed that the purified protein was of the correct size and approximately 90% pure. Western blot verified the identity of the Neprilysin domain.

### Example 11

### Neprilysin Enzyme Activity FRET-Assay

The Neprilysin enzymatic activity was determined in a fluorescence resonance energy transfer (FRET) assay. Recombinant Human Neprilysin (R&D Systems), culture medium from Neprilysin or Fc-Neprilysin producing cells (AZ Sodertalje) or purified Neprilysin or Fc-Neprilysin was added into 96-well plate containing 10 μM of fluorogenic peptide substrate V—Mca-Arg-Pro-Gly-Phe-Ser-Ala-Phe-Lys(Dnp)-OH (R&D Systems). The final concentration of the control recombinant human Neprilysin was 0.1 or 0.25 μg/ml. 10 μM of Neprilysin inhibitor phosphoramidone (BIOMOL) was added into some wells in order to control the specificity of the signal in the assay and verify the specific Neprilysin activity. Following addition of all components to wells, plate was immediately placed into a fluorescent plate reader (Ascent) and signal was recorded for every minute for 20 minutes at the excitation 340 nm and emission 405 nm. The activity of enzyme was evaluated by calculating the velocity of reaction−Slope coefficient=ΣΔRFU/Δt. In order to compare the specific activity of the commercial recombinant Neprilysin and Fc-Neprilysin fusion protein, we introduced the specific activity coefficient, which is calculated according to this formula: Specific activity=slope coefficient/pmol of Neprilysin or monomer of Fc-Neprilysin in assay.

### Example 12

### IDE and ECE1 Enzyme Activity FRET-Assay

The enzymatic activity is determined in a fluorescence resonance energy transfer (FRET) assay. Recombinant enzyme without Fc domain (commercial or in-house produced), culture medium (from Fc-IDE or Fc-ECE1-producing cells) or purified protein (Fc-IDE or Fc-ECE1) is added into 96-well plate containing 10 μM of fluorogenic peptide substrate V—Mca-Arg-Pro-Gly-Phe-Ser-Ala-Phe-Lys(Dnp)-OH (R&D Systems). Following addition of all components to wells, plate is immediately placed into a fluorescent plate reader (Ascent) and signal is recorded for every minute for 20 minutes at the excitation 340 nm and emission 405 nm. The activity of enzyme is evaluated by calculating the velocity of reaction−Slope coefficient=ΣΔRFU/Δt. In order to compare the specific activity of the control (commercial recombinant enzyme) the specific activity coefficient is calculated according to this formula: Specific activity=slope coefficient/pmol of enzymatic domain in assay.

### Example 13

### Measurement of Neprilysin and Fc-Neprilysin Concentration in Cell Culture Supernatant

Neprilysin concentration in cell culture supernatant was measured using Gyros™ Bioaffy™ CD microlaboratory method and Gyrolab Workstation LIF equipment (Gyros AB, Sweden). Samples from different cell cultures were diluted in Standard Diluent (Gyros AB) and placed into Thermo-Fast© 96-well PCR plate (Abgene, UK). Monoclonal mouse biotinylated anti-human Neprilysin antibody (Serotec) was used as a capturing reagent (final concentration 0.05 mg/ml) and polyclonal goat anti-human Neprilysin antibody (R&D Systems) labeled with Alexa Fluor 647 dye (Molecular Probes) served as a detection antibody (final concentration 100 nM) for measurement of Neprilysin concentrations. Commercial recombinant Neprilysin (R&D Systems) was used as a standard in a concentration range from 10 ng/ml to 10000 ng/ml in order to construct a standard curve. Polyclonal biotinylated anti-human Neprilysin antibody (R&D Systems) was used as a capturing antibody, while polyclonal goat anti-human IgG antibody (Molecular Probes) labeled with Alexa Fluor 647 dye (Molecular probes) was used as a detection antibody for Fc-Neprilysin construct detection. In-house produced and purified Fc-Neprilysin fusion protein served as a standard in a concentration range from 10 ng/ml to 10000 ng/ml. Standards, capturing and detection antibodies were placed to Thermo-Fast© 96-well PCR plate (Abgene). Both plates as well as Gyrolab Bioaffy™ CD were placed into Gyrolab Workstation LIF instrument and concentration measurement performed according to the manufacturers protocol using Gyrolab Bioaffy™ Software Package Version 1.8 (Gyros AB).

### Example 14

### Measurement of IDE, ECE1, Fc-IDE and Fc-ECE1 Concentration in Cell Culture Supernatant

Protein concentration in cell culture supernatant is measured using Gyros™ Bioaffy™ CD microlaboratory method and Gyrolab Workstation LIF equipment (Gyros AB, Sweden). Samples from different cell culture conditions are diluted in Standard Diluent (Gyros AB) and placed into Thermo-Fast© 96-well PCR plate (Abgene, UK). Biotinylated IDE or ECE1-specific antibodies are used as a capturing reagent and Alexa Fluor 647 dye (Molecular Probes) labelled IDE or ECE1-specific antibodies are used as detection antibodies. Commercial or in-house produced recombinant IDE and ECE1 is used to construct a standard curve. When measuring Fc-IDE or Fc-ECE1 concentration, the difference is that a polyclonal goat anti-human IgG antibody (Molecular Probes) labeled with Alexa Fluor 647 dye (Molecular probes) is used as a detection antibody. Standards, capturing and detection antibodies are placed to Thermo-Fast© 96-well PCR plate (Abgene). Both plates as well as Gyrolab Bioaffy™ CD are placed into Gyrolab Workstation LIF instrument and concentration measurement performed according to the manufacturers protocol using Gyrolab Bioaffy™ Software Package Version 1.8 (Gyros AB).

### Example 15

### Degradation of Amyloid β Peptide by Fc-Neprilysin and Neprilysin in Buffer

The goal of this experiment was to demonstrate that Fc-Neprilysin is capable to degrade amyloid β 1′-40 peptide. The assay is measuring the remaining amyloid β 1-40 peptide (Bachem) concentration following its incubation in the presence of Neprilysin (R&D Systems) or Fc-Neprilysin with or without Neprilysin inhibitor. 100 μl of reaction mixture containing amyloid β 1-40 peptide (final concentrations, 300, 30 or 3 nM) and/or Neprilysin (2.4 μg/ml), and/or Fc-Neprilysin construct (2.4 μg/ml), and/or Phosphoramidone (10 μM) was incubated in a round bottom 96-well polypropylene plate at 37° C. for 2.5 hours. Following incubation, 10 μl of reaction mixture was transferred into Thermo-Fast© 96-well PCR plate (Abgene, UK) containing 10 μl of Standard Diluent (Gyros AB). Amyloid β 1-40 concentration was determined using Gyrolab Workstation LIF system. Biotinylated anti-amyloid β antibodies (6E10; final concentration 50 μg/ml; Signet) were used as capturing antibodies and polyclonal anti-human amyloid β antibodies (44-348; Biosource) labeled with Alexa Fluor 647 dye (Molecular probes) were used as detection antibodies. Amyloid β 1-40 peptide concentration measurement performed according to the manufacturers protocol using Gyrolab Bioaffy™ Software Package Version 1.8 (Gyros AB). Amyloid β 1-40 peptide degradation by Neprilysin was calculated as a percentage of Amyloid β 1-40 peptide left after incubation in the presence of Neprilysin compared to the amyloid β 1-40 peptide concentration in the absence of Neprilysin. Recombinant human Neprilysin at the concentration of 2.4 μg/ml after 2.5 hours incubation at 37° C. degraded 64% of Amyloid β 1-40 peptide (300 nM). In-house produced Fc-Neprilysin construct at approximately the same concentration (2.4 μg/ml) degraded 50% (batch 1) and 42% (batch 2) of amyloid β1-40 peptide (300 nM). The specific Neprilysin activity was almost completely abolished in the presence of 10 μM Phosphoramidone (FIG. 1). This example shows that Fc-Neprilysin effectively degrades the amyloid β 1-40 peptide.

### Example 16

### Degradation of Amyloid β Peptide by IDE, ECE1, Fc-IDE and Fc-ECE1 in Buffer

The goal of this experiment is to demonstrate that Fc-IDE and Fc-ECE1 is capable to degrade amyloid β1-40 peptide. The assay is measuring the remaining amyloid β 1-40 peptide (Bachem) concentration following its incubation in the presence of enzyme (Fc-IDE or Fc-ECE1). 100 μl of reaction mixture containing amyloid β 1-40 peptide (final concentrations, 300, 30 or 3 nM), Fc-IDE or Fc-ECE1 is incubated at 37° C. for 2.5 hours. Following incubation, 10 μl of reaction mixture is transferred into Thermo-Fast© 96-well PCR plate (Abgene, UK) containing 101 of Standard Diluent (Gyros AB). Amyloid β 1-40 concentration is determined using Gyrolab Workstation LIF system. Biotinylated anti-amyloid β antibodies (6E10; final concentration 50 μg/ml; Signet) are used as capturing antibodies and polyclonal anti-human amyloid β antibodies (44-348; Biosource) labeled with Alexa Fluor 647 dye (Molecular probes) are used as detection antibodies. Amyloid β 1-40 peptide degradation by Neprilysin is calculated as a percentage of Amyloid β 1-40 peptide left after incubation in the presence of enzymes compared to the amyloid β 1-40 peptide concentration in the absence of enzymes.

### Example 17

### Degradation of Amyloid β Peptide 1-40 and Amyloid β Peptide 1-42 in Guinea Pig Plasma by Fc-Neprilysin

Degradation of amyloid β peptide 1-40 (Aβ40) and amyloid β peptide 1-42 (Aβ42) by neprilysin was investigated using heparinized plasma from male Dunkin Hartley guinea pigs, weighing 250-300 g (HBLidkoping ka). Blood was withdrawn from anaesthetized guinea pigs by heart puncture. The blood were collected into prechilled heparin-plasma tubes and centrifuged for 10 min at 4° C. at 3000×g within 20 minutes of sampling. Plasma samples were transferred to pre-chilled polypropylene tubes and immediately frozen on dry ice and stored at −70° C. prior to use. The experiments were performed on a pool of plasma from seven guinea pigs. His-Fc-Nep (6 μg/ml or 208 μg/ml) or 5 μg/ml recombinant human Neprilysin (R&D systems) with corresponding vehicles (50 mM Tris-HCl, 150 mM NaCl pH 7.5 or 25 mM Tris-HCl, 0.1 M NaCl pH 8.0) were incubated with a pool of plasma in presence or absence of 10 μM phosphoramidon (BIOMOL) at 37° C. for 0 and 4 h. A final concentration of 4.7 mM EDTA was added into the tubes before the amount of Aβ40 and Aβ42 was analysed using a commercial ELISA kit obtained from Biosource (Aβ1-40) or Innogenetics (Aβ1-42).

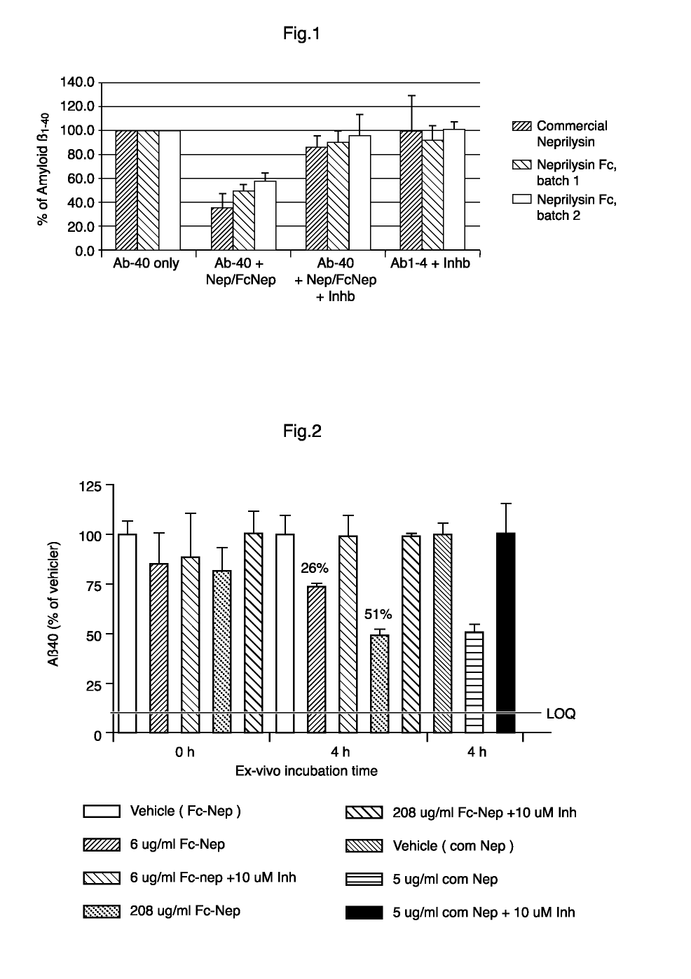

Ex-vivo incubation of 4 hours in 37° C. with guinea pig plasma and 6 μg/ml or 208 μg/ml His-Fc-Nep resulted in reduction of Aβ40 with 26% and 51%, respectively, compared to vehicle. Commercial human recombinant neprilysin (5 μg/ml) degraded Aβ40 with 49% compared to vehicle. The Aβ40 levels were unaffected after addition of 10 μM phosphoramidon (FIG. 2).

Aβ42 levels in guinea pig plasma were reduced more than 57%, compared to vehicle when incubated either with 208 μg/ml His-Fc-Nep or 5 μg/ml Neprilysin (R&D Systems). The reduction of Aβ42 was not inhibited by phosphoramidon when combined with 208 μg/ml of the His-Fc-Nep. There was no degradation in Aβ42 with the low concentration of His-Fc-Nep (FIG. 3).

### Example 18

### Degradation of Amyloid β Peptide 1-40 in Human Plasma by Fc-Neprilysin

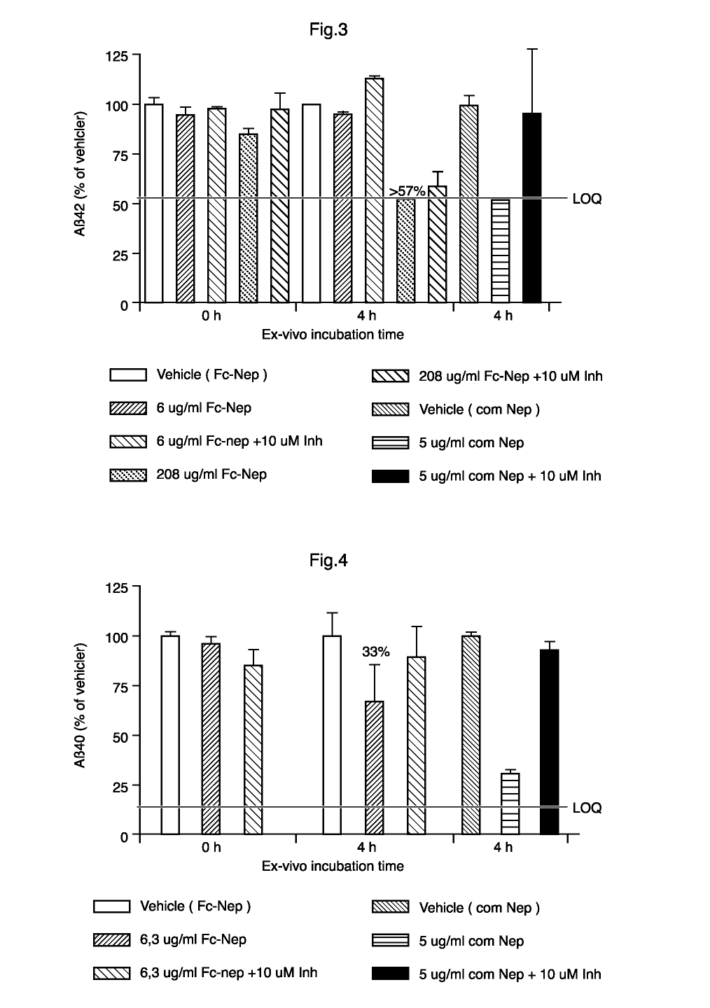

Blood from eight individuals (5 females and 3 males) were collected into pre-chilled heparin-plasma tubes at the healthcare centre (AstraZeneca) at two different time points. Plasma was prepared by centrifugation for 20 min at 4° C. at 2500×g within 30 minutes of sampling. Plasma samples were transferred to pre-chilled polypropylene tubes and immediately frozen and stored at −70° C. prior to use. His-Fc-Nep (6 μg/ml) or 5 μg/ml recombinant human Neprilysin (R&D systems) with corresponding vehicles (50 mM Tris-HCl, 150 mM NaCl pH 7.5 or 25 mM Tris-HCl, 0.1 M NaCl pH 8.0) in presence or absence of 10 μM phosphoramidon was incubated with a pool of plasma at 37° C. for 0 and 4 h. A final concentration of 4.7 mM EDTA was added into the tubes before the amount of Aβ40 was analysed using a commercial ELISA kit obtained from Biosource. His-Fc-Nep (6 μg/ml) and commercial human recombinant neprilysin (5 μg/ml) degraded Aβ40 with 33% and 70%, respectively, compared to vehicle after 4 hours incubation at 37° C. The Aβ40 levels were unaffected after addition of 10 μM phosphoramidon (FIG. 4).

### Example 19

### Degradation of Amyloid β Peptide 1-40 and Amyloid β Peptide 1-42 in Guinea Pig Plasma by in-House Produced Fc-Nep (In Vivo Studies)

In vivo studies in guinea pigs are performed in order to test the in vivo efficacy of in-house produced Fc-Nep. The read-out is plasma Aβ levels and plasma drug concentration. The γ-secretase inhibitor, AZ10420130 (M550426) is used as reference (positive control for reduction of plasma Aβ levels).

The guinea-pigs (Male Dunkin Hartley Guinea pigs, 250-300 g) are weighed and i.v. administrated with a single dose. The aim is that the dose should give the plasma exposures 0, 5, and 20 μg/ml at termination. Observations of the animal health are made during the whole experiment. 8 animals are included in each time point and each time points has its own vehicle group. The animals are anaesthetized with Isoflurane and blood is sampled by heart puncture. For information about blood sample handling and analysis of Aβ1-40 or Aβ1-42 (See Example 23). All plasma samples will be sent for PK studies to determine drug exposure (For method description, see Example 20).

### Example 20

### Pharmacokinetics of Fc-Nep and Neprilysin Only

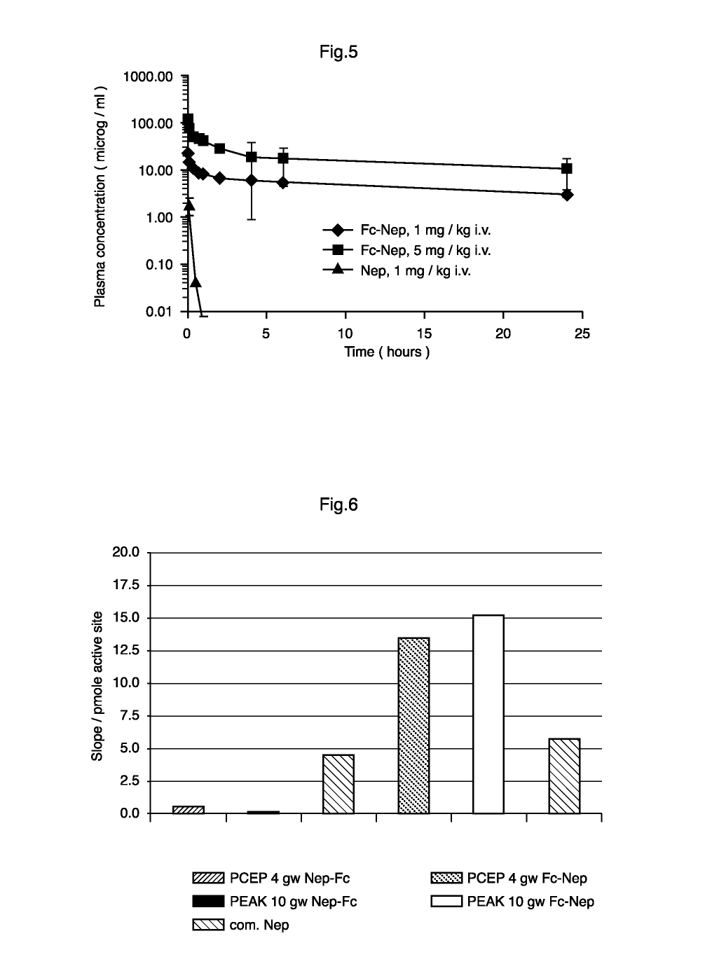

The Fc-Nep fusion protein was developed to improve the pharmacokinetic entities of neprilysin with the specific aims to reduce clearance and improve half-life. To test this we have administrated a single i.v. dose of either 1 mg/kg commercial neprilysin or 1 alternatively 5 mg/kg in-house produced Fc-Nep to mice. At set times after dosing blood samples were drawn from the tail vein or by heart puncture at termination. Upon sampling into tubes containing EDTA the aliquots were put on ice. Plasma was prepared by centrifugation within 15 minutes of sampling (typically 1500 g at 4° C. for 10 min) and immediately frozen. Plasma concentrations of Fc-Nep and neprilysin were determined via immunoassays using either anti-Nep for commercial neprilysin or anti-human IgG for Fc-Nep as capture antibodies while both substances were detected via an anti-Nep antibody. Pharmacokinetic parameters are calculated using a software package (WinNonlin, Pharsight Corporation, USA) and in this example experiment the calculated half-life had increased from about 5 minutes for Nep to about 20 hours for Fc-Nep. The results are shown in FIG. 5.

### Example 21

### Comparison of the Enzymatic Activity of Neprilysin-Fc and Fc-Neprilysin in Cell Media Using Enzyme Activity Fret-Assay

In order to compare C-terminal fusion of Fc to Neprilysin and N-terminal fusion of Fc to Neprilysin, both proteins (Fc-Neprilysin and Neprilysin-Fc) was produced according to Example 4 and purified as described in Example 8. The enzymatic activity of the protein in cell media was determined in a fluorescence resonance energy transfer (FRET) assay. Recombinant Human Neprilysin (R&D Systems), culture medium from Fc-Neprilysin producing cells and from Neprilysin-Fc producing cells was added into 96-well plate containing 10 μM of fluorogenic peptide substrate V—Mca-Arg-Pro-Gly-Phe-Ser-Ala-Phe-Lys(Dnp)-OH (R&D Systems). The final concentration of the control recombinant human Neprilysin was 0.1 or 0.25 μg/ml. Following addition of all components to wells, plate was immediately placed into a fluorescent plate reader (Ascent) and signal was recorded for every minute for 20 minutes at the excitation 340 nm and emission 405 nm. The activity of enzyme was evaluated by calculating the velocity of reaction−Slope coefficient=ΣΔRFU/Δt. In order to compare the specific activity of the commercial recombinant Neprilysin, Neprilysin-Fc fusion protein and Fc-Neprilysin fusion protein, we introduced the specific activity coefficient, which is calculated according to this formula: Specific activity=slope coefficient/pmol of Neprilysin or monomer of fusion protein in assay. The results (shown in FIG. 6) show that the expression of Nep-Fc resulted in a very low specific activity (0.1 for expression with pCEP4 vector and 0.55 for expression with pEAK 10 vector) but the expression of Fc-Nep resulted in a much higher specific activity (13.4 for expression with pCEP4 vector and 15.2 for expression with pEAK10 vector).

### Example 22

### Comparison of the Enzymatic Activity of Purified Neprilysin-Fc and Fc-Neprilysin using Enzyme Activity FRET-Assay

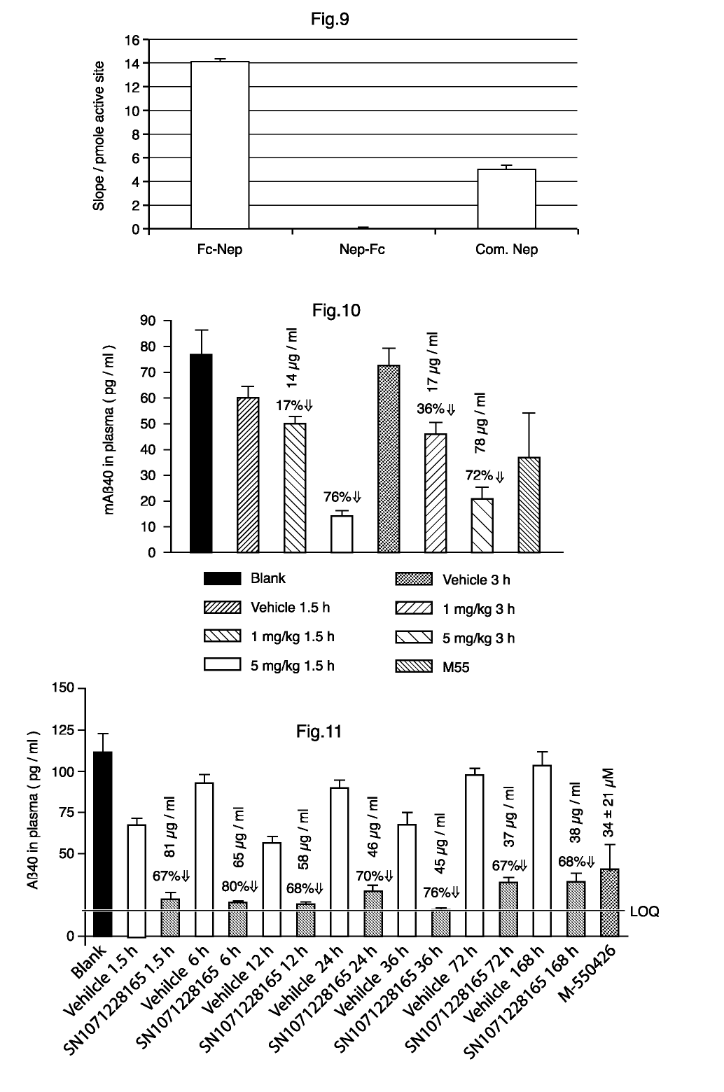

In order to compare C-terminal fusion of Fc to Neprilysin and N-terminal fusion of Fc to Neprilysin, both proteins (Fc-Neprilysin and Neprilysin-Fc) was produced according to Example 4 and purified as described in Example 8. The Neprilysin enzymatic activity was determined in a fluorescence resonance energy transfer (FRET) assay. Recombinant Human Neprilysin (R&D Systems), purified Neprilysin-Fc protein and purified Fc-Neprilysin was added into 96-well plate containing 10 μM of fluorogenic peptide substrate V—Mca-Arg-Pro-Gly-Phe-Ser-Ala-Phe-Lys(Dnp)-OH (R&D Systems). Following addition of all components to wells, plate was immediately placed into a fluorescent plate reader (Ascent) and signal was recorded for every minute for 20 minutes at the excitation 340 nm and emission 405 nm. The activity of enzyme was evaluated by calculating the velocity of reaction−Slope coefficient=ΣΔRFU/Δt. In order to compare the specific activity of the commercial recombinant Neprilysin, Neprilysin-Fc fusion protein and Fc-Neprilysin fusion protein, we introduced the specific activity coefficient, which was calculated according to this formula: Specific activity=slope coefficient/pmol of Neprilysin or monomer of Neprilysin-Fc in assay. The results (shown in FIG. 9) show that the specific activity of the purified fusion protein Nep-Fc was very low (0.001) but the specific activity of the purified fusion protein Fc-Nep was much higher (14.1).

### Example 23

### Treatment with Fc-Neprilysin on Soluble Aβ Levels in Plasma in APP_{SWE}-Transgenic Mice

The objective with this study was to evaluate the time and dose-response effect of Fc-Nep in plasma of female APP_{SWE}-tg mice after acute intraveneous treatment. The specific purpose is to find an effect on plasma Aβ₄₀ and Aβ₄₂. The γ-secretase inhibitor M-550426 is included as a reference compound.

25-31 weeks old female APP_{SWE}-transgenic mice (10 mice/group) received vehicle or the Fc-Nep at 1 or 5 mg/kg as a single intravenous injections. As a reference compound, 300 μmol/kg of the γ-secretase inhibitor M-550426 was used and these animals was treated in 3 hours (4 mice). A blank group (4 untreated mice) was also induced in the study. Blood was sampled from vehicle- and compound-treated animals at 1.5 and 3 hours after dose. Blood was withdrawn from anaesthetized mice by heart puncture into pre-chilled microtainer tubes containing EDTA. Blood samples were immediately put on ice prior to centrifugation. Plasma was prepared by centrifugation for 10 minutes at approximately 3000×g at +4° C. within 20 minutes from sampling. After blood sampling, mice were terminated. Aβ40 and Aβ42 levels in plasma were analyzed by commercial ELISA kit obtained from Biosource and Innogenetics, respectively.

The concentrations of Fc-Nep in plasma and in the formulations were assayed according to the procedures described in Example 20. The exposure in plasma was analysed in samples from non-treated animals (blank) and in samples from animals treated with M550426.

### Results

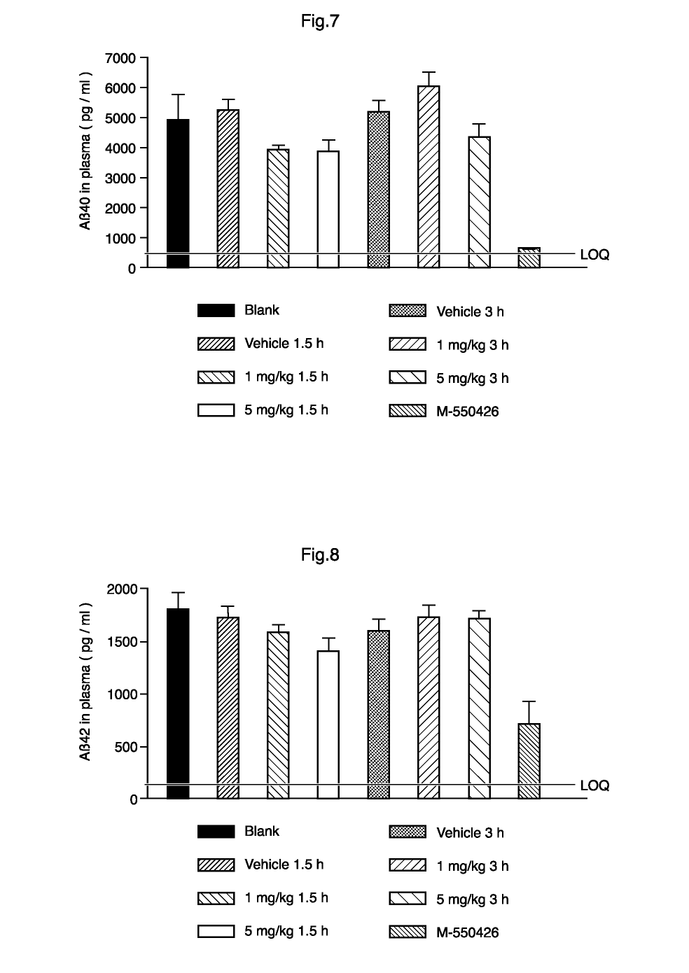

The Fc-Nep significantly reduced the level of soluble Aβ40 with approximately 20% compared to vehicle (P<0.05) in plasma at 1.5 hours after 1 or 5 mg/kg i.v. injection dose, but not at 3 hours after dose in APP_{swe} transgenic mice. The mean plasma exposure of Fc-Nep at 1 and 5 mg/kg at 1.5 hours was 9.8 and 33.6 μg/ml, respectively. No significant changes in Aβ40 was seen after 3 hours although the Fc-Nep plasma exposure at 1 and 5 mg/kg was 7.6 and 27.3 μg/ml, respectively. As expected, decreased levels of Aβ40 was observed in plasma after treatment with the positive control, γ-secretase inhibitor M550426. The mean plasma exposure of M550426 at 3 hours after dose was 33.5 μM in mice receiving 300 μmol/kg (FIG. 7).

The level of Aβ42 in plasma after 1.5 hours treatment with 5 mg/kg Fc-Nep was reduced by approximately 20% compared to vehicle (P<0.05) No significant change was seen after 1.5 hours of 1 mg/kg administration. No significant changes in Aβ42 was seen in any of the doses after 3 hours although the Fc-Nep plasma exposure at 1 and 5 mg/kg was 7.6 and 27.3 μg/ml, respectively. Decreased levels of Aβ42 was observed in plasma after treatment with the positive control, γ-secretase inhibitor M550426. The mean plasma exposure of M550426 at 3 hours after dose was 33.5 μM in mice receiving 300 μmol/kg (FIG. 8).

### Example 24

### Treatment with hFc-Nep and the Effect on Soluble Aβ Levels in Plasma in C57BL/6 Mice (Time- and Dose Response Study: 1.5 & 3 Hours)

The objective with this study was to evaluate the time and dose-response effect of hFc-Nep in plasma of female C57BL/6 mice after an acute treatment. The specific purpose is to find an effect on plasma Aβ40 and to correlate effect to exposure level of hFc-Nep in plasma. The γ-secretase inhibitor M-550426 is included as a positive control.

13 weeks old female C57BL/6 mice (10 mice/group) received vehicle or hFc-Nep at 1 or 5 mg/kg as a single intravenous injection. M-550426 was administrated per orally at 300 μmol/kg 3 hours before termination. A blank group was also included in the study.

Blood was sampled from vehicle- and compound-treated animals at 1.5 and 3 hours after dose. Blood was withdrawn from anaesthetized mice by heart puncture into pre-chilled microtainer tubes containing EDTA. Blood samples were immediately put on ice prior to centrifugation. Plasma was prepared by centrifugation for 10 minutes at approximately 3000×g at +4° C. within 20 minutes from sampling. After blood sampling, mice were terminated. Observations of the animal health were made during the whole experiment revealing no overt adverse effects. Mouse Aβ40 levels in plasma were analysed by commercial ELISA kit obtained from Biosource. The concentrations of Fc-Nep in plasma and in the formulations were assayed according to the procedures described in Example 27.

### Results

The results showed that mouse Aβ40 is significantly reduced by treatment with hFc-Nep in a dose-dependent manner both after 1.5 and 3 hours in C57BL/6 mice. After 1.5 hours, a reduction of Aβ40 of 17% was seen at 1 mg/kg dose (p=0.1638) and 76% reduction at 5 mg/kg dose (p<0.0001) compared to vehicle. The mean plasma exposure of hFc-Nep at 1 and 5 mg/kg at 1.5 hours was 14 and 89 μg/ml, respectively. After 3 hours, Aβ40 was significantly reduced with 36% at 1 mg/kg dose (p<0.005) and with 72% at 5 mg/kg dose (p<0.0001) compared to vehicle. The mean plasma exposure of hFc-Nep at 1 and 5 mg/kg at 3 hours was 17 and 78 μg/ml, respectively. As expected, decreased levels of Aβ40 were also observed in plasma after treatment with the positive control, γ-secretase inhibitor M-550426. The mean plasma exposure of M-550426 at 3 hours after dose was 42 μM in mice receiving 300 μmol/kg (FIG. 10).

### Example 25

### Time-Response Relationship Using hFc-Nep Given as a Single Dose Via Intravenous Injection to C57BL/6 Mice

The objective of this study was to evaluate the time-response relationship of the hFc-Nep in plasma of female C57BL/6 mice after a single dose. The specific purpose is to find how long the reducing effect of hFc-Nep stays in the plasma, and to correlate the effect to the level of exposure of test compound in plasma. The γ-secretase inhibitor M-550426 is included as a positive compound.

20-21 weeks old female C57BL/6 mice (8 mice/group) received vehicle or hFc-Nep at 5 mg/kg as a single intravenous injection and Aβ40 was analysed at different time points after injection (between 1.5-168 hours, i.e., up to 1 week). The γ-secretase inhibitor M-550426 was given per orally and the animals were treated for 3 hours. A blank group was also included in the study. Observations of the animal health were made during the whole experiment revealing no overt adverse effects. Blood collection, plasma processing and measurement of mouse Aβ40 levels in plasma were basically as described in Example 27.

### Results

The results (FIG. 11) showed that plasma Aβ40 is significantly reduced after 1.5-168 hours' treatment of hFc-Nep when given as a single intravenous injection to C57BL/6 mice. The Aβ40 reduction was persistent (between 67-80% compared to vehicle) at all time points (1.5, 6, 12, 24, 36, 72 and 168 hours). The mean plasma exposure of hFc-Nep at 5 mg/kg was 87 μg/ml at 1.5 hours and was slowly reduced to a level of 38 μg/ml after 1 week (168 hours). These data show that the half-life of Fc-Nep in mice is considerably long. As expected, decreased levels of Aβ40 was observed in plasma after treatment with the positive control, 7 secretase inhibitor M550426. The mean plasma exposure of M-550426 at 3 hours after dose was 34 μM in mice receiving 300 μmol/kg (FIG. 11).

### Example 26

### Time-Response Relationship Using Mouse Fc-Nep Given as a Single Dose Via Intravenous Injection to APP_{SWE}-Tg Mice and C57BL/6

The objective of this study was to evaluate the time-response relationship of the mouse version of the Fc-Nep (mFc-Nep, SEQ ID NO 14) in plasma of female APP_{SWE}-tg mice and C57BL/6 mice after a single dose. The specific purpose is to find out how long the reducing effect of mFc-Nep on Aβ stays in the plasma, and to correlate the effect to the level of exposure of test compound in plasma. The γ-secretase inhibitor M-550426 is included as a positive compound.

21-23 weeks old female APP_{SWE}-tg mice and 24 weeks old female C57BL/6 mice (6 mice/group) received vehicle or mFc-Nep at 5 or 25 mg/kg as a single intravenous injection and Aβ40 was analysed at different time points after injection (between 1.5-336 hours, i.e., up to 2 weeks). M-550426 was administrated per orally at 300 μmol/kg 3 hours before termination. For both mouse models, APP_{SWE}-tg and C57BL/6, a positive control and blank groups were included. The following groups were included for the APP_{SWE}-tg mice: 25 mg/kg: 1.5, 72, 168 and 336 hours; 5 mg/kg): 336 hours (2 weeks). For C57BL/6 mice: 25 mg/kg: 168 and 336 hours; 5 mg/kg): 1.5, 168 and 336 hours. Observations of the animal health were made during the whole experiment revealing no overt adverse effects. Blood collection and plasma processing were basically as described in Example 25 The analysis of mouse Aβ40 levels in plasma of C57BL6 mice was as described in Example 25 The analysis of human Aβ40 and Aβ42 levels in plasma of APP_{SWE}-tg mice was as described in Example 25 (as described in the last APP-tg study).

### Results

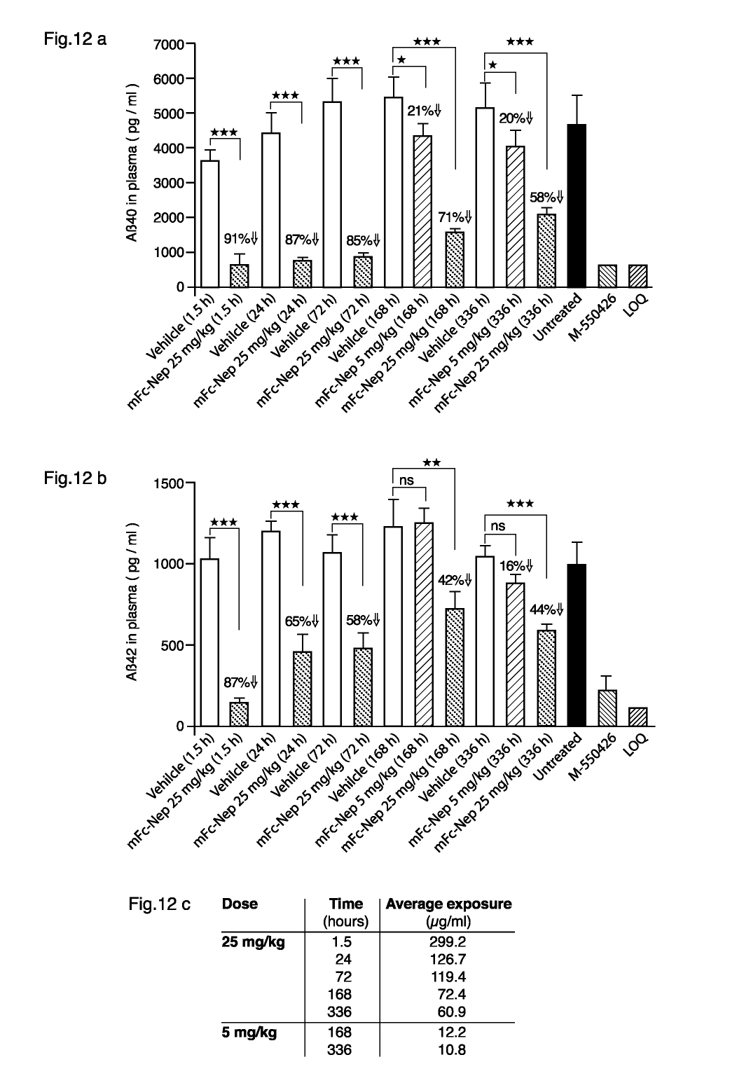

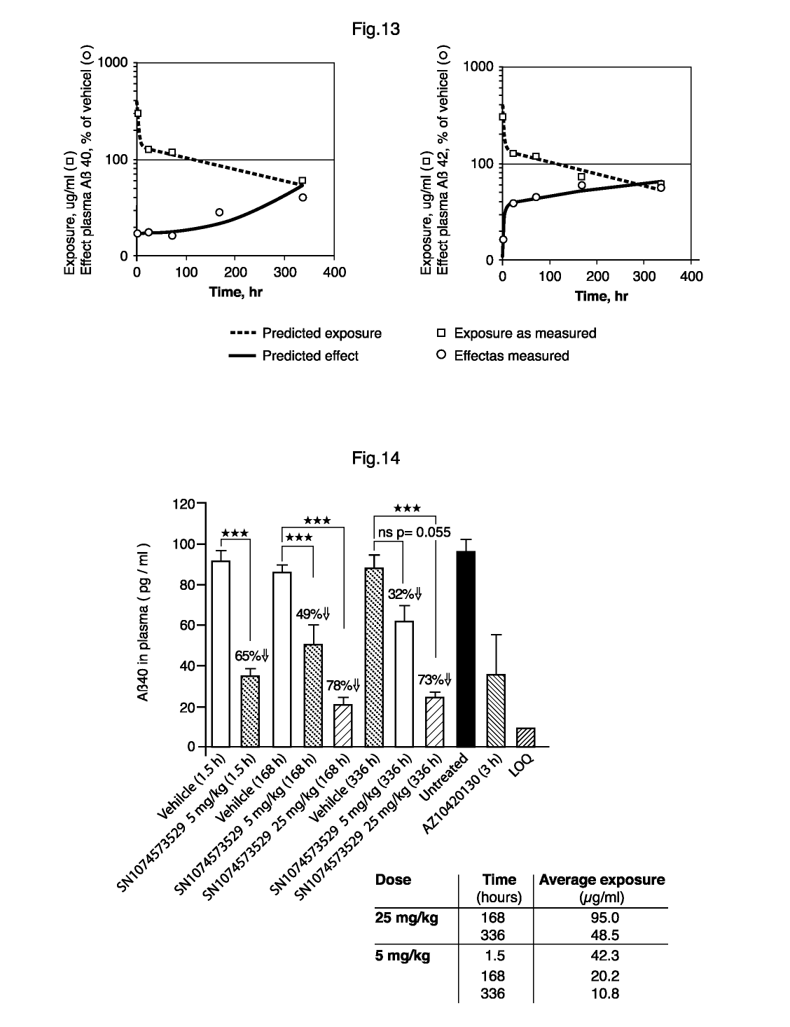

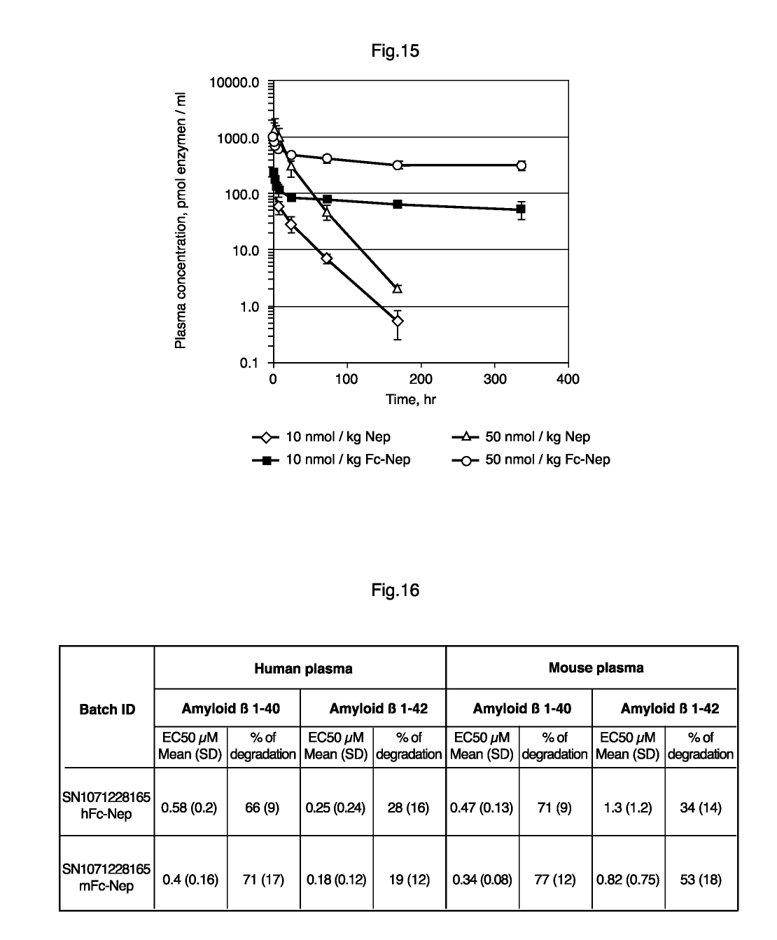

In APP_{SWE}-transgenic mice, mFc-Nep significantly reduced human Aβ40 and Aβ42 in plasma at all time points after a single administration of 25 mg/kg (FIG. 12, a and b). After 1.5 hours, the Aβ levels are 91% and 87% for Aβ40 and Aβ42, respectively, when compared to vehicle and the Aβ levels gradually increased when the exposure is decreased. After two weeks (336 hours), the Aβ levels are 58% and 44% for Aβ40 and Aβ42, respectively, when compared to vehicle. After two weeks, the exposure after a single intravenous injection of 25 mg/ml mFc-Nep has reduced from 299 μg/ml (1.5 hours) down to 60 μg/ml (336 hours) (FIG. 12, c). For 168 and 336 hours, an additional group of animals were used that was given 5 mg/kg. As shown in FIG. 12, a and b, Aβ is degraded in a dose-dependent manner at those time points for both Aβ40 and Aβ42. The plasma efficacy effects of both Aβ40 and Aβ42 are inversely correlated to the plasma exposure of mFc-Nep (FIG. 13). These results indicate that mFc-Nep's Aβ degrading effect is greater for Aβ40 than for Aβ42.

In C57BL/6 mice, mFc-Nep significantly reduce mouse Aβ40 in plasma in at both 5 and 25 mg/kg at all time points (1.5, 168 and 336 hours) (FIG. 14). At 168 and 336 hours, both 5 and 25 mg/kg was analysed and the Aβ40 effects are shown to be dose-dependent. After 2 weeks, a single injection (336 hours) of 25 mg/kg mFc-Nep, significantly reduce the mouse Aβ40 levels in plasma by 73% compared to vehicle. The plasma exposure at this time point was 48 μg/ml and mFc-Nep thereby show to have a long plasma half-life.

### Example 27

### Pharmacokinetics of Fc-Nep and in-House Produced Neprilysin

Pharmacokinetic studies were repeated using different batches of Fc-Nepa and Nep and different PK profile was obtained. Most important is the significant prologantion of plasma half-life of the compound including the Fc-part for an IgG.

The Fc-Nep fusion protein was developed to improve the pharmacokinetic entities of neprilysin with the specific aims to reduce clearance and improve half-life. To test this we have administrated a single i.v. dose of 10 or 50 nmol enzyme/kg body weight neprilysin (Nep) or Fc-Nep (1 and 5 mg/kg) to mice. At set times the dose blood samples were drawn from the tail vein or by heart puncture at termination. Upon sampling into tubes containing EDTA the aliquots were put on ice. Plasma was prepared by centrifugation within 15 minutes of sampling (typically 1500 g at 4° C. for 10 min) and immediately frozen. Plasma concentrations of Nep and Fc-Nep were determined via immunoassays using either anti-Nep for Nep or anti-human IgG for Fc-Nep as capture antibodies while both substances were detected via an anti-Nep antibody. Pharmacokinetic parameters are calculated using a software package (WinNonlin, Pharsight Corporation, USA) and in this example experiment the calculated half-life had increased from about 1 day for Nep to about 2.5 weeks for Fc-Nep. The results are shown in FIG. 15.

### Example 28

### Degradation of Amyloid β Peptide 1-40, 1-42 in Human and APP_{swe}-Tg Mouse Plasma by Human or Mouse Fc-Neprilysin

Blood from twelve individuals (6 females and 6 males) were collected into pre-chilled heparin-plasma tubes at the healthcare centre (AstraZeneca) at three different time points. Plasma was prepared by centrifugation at 2500×g for 20 min at 4° C. Plasma samples were collected and transferred to pre-chilled polypropylene tubes and immediately frozen and stored at −70° C. prior to use. Plasma was thawed and pooled from 12 individuals just before the experiment. Aβ 1-40 and 1-42 in plasma pool was degraded by human Fc-Nep or mouse Fc-Nep with corresponding vehicles (50 mM Tris-HCl, 150 mM NaCl pH 7.5). The following final concentrations of the Fc-Nep constructs were used, 100, 32, 10, 3.2, 1, 0.3, 0.1 and 0 pg/ml and the degradation occurred at room temperature for 1 hour while shaking on an orbital shaker. The enzymatic reaction was stopped by adding Phosphoramidone (10 μM final concentration). Concentration of amyloid β 1-40 in human plasma pool was measured using ELISA kit (Biosource; KHB3481) according to the manufacturers instructions.

A final concentration of 4.7 mM EDTA was added to the tubes before the concentration of Aβ42 was analyzed using ELISA kit Innotest® β-Amyloid₁₋₄₂ (Innogenetics, lot#177462, ref#80177) according to the manufacturers instructions.

The highest concentration (100 μg/ml) of human Fc-Nep and mouse Fc-Nep degraded human plasma amyloid β 1-40 by 66% and 71%, respectively and Aβ 1-42 by 28% and 19%, respectively, as compared to plasma without Fc-Nep treatment. EC₅₀ values of degradation by human Fc-Nep and mouse Fc-Nep was for human Aβ 1-40 0.58 μM and 0.40 μM, respectively and for Aβ 1-42 0.25 μM and 0.18 μM respectively. Results are summarized in FIG. 16.

Mouse plasma collected from 9 animals was stored at −70° C. Plasma was thawed and pooled just before the experiment. Aβ 1-40 and 1-42 in plasma pool was degraded by human Fc-Nep or mouse Fc-Nep with corresponding vehicles (50 mM Tris-HCl, 150 mM NaCl pH 7.5). The following final concentrations of the Fc-Nep constructs were used, 100, 32, 10, 3.2, 1, 0.3, 0.1 and 0 pg/ml and the degradation occurred at room temperature for 1 hour while shaking on an orbital shaker. The enzymatic reaction was stopped by adding Phosphoramidone (10 μM final concentration).

After degradation, before concentration of amyloid β 1-40 in tg-mouse plasma pool was measured using ELISA kit (Biosource; KHB3481), the plasma samples were diluted 20 times in standard diluent buffer, according to manufacturers instructions.

After degradation, a final concentration of 4.7 mM EDTA was added to the tg-mouse plasma tubes and the plasma samples were diluted 3 times in sample diluent, before the concentration of Aβ42 was analyzed using ELISA kit Innotest® β-Amyloid₁₋₄₂ (Innogenetics, lot#177462, ref#80177) according to the manufacturers instructions. The highest concentration (100 μg/ml) of human Fc-Nep and mouse Fc-Nep degraded human plasma amyloid β 1-40 by 71% and 77%, respectively and Aβ 1-42 by 34% and 53%, respectively, as compared to plasma without Fc-Nep treatment. EC₅₀ values of degradation by human Fc-Nep and mouse Fc-Nep was for human Aβ 1-40 0.47 μM and 0.34 μM, respectively and for Aβ 1-42 1.3 μM and 0.82 μM respectively. Results are summarized in FIG. 16.

1. A fusion protein having the formula M-A, capable of degrading amyloid beta peptide at one or more cleavage sites in said amyloid beta peptide amino acid sequence, wherein M is a protein component that prolongs the half-life of the fusion protein, and A is a protein component that cleaves the amyloid beta peptide, wherein said M protein component is covalently connected to the N-terminus part of the A protein component.

2. The fusion protein according to claim 1, wherein A is a protease.

3. The fusion protein according to claim 1, wherein A is human Neprilysin.

4. The fusion protein according to claim 3, wherein said Neprilysin is extracellular Neprilysin.

5. The extracellular Neprilysin according to claim 4, comprising an amino acid sequence according to any one of SEQ ID NO. 1, 2, 3 or 4.

6. The fusion protein according to claim 1, wherein A is insulin-degrading enzyme.

7. The fusion protein according to claim 1, wherein A is endothelin-converting enzyme 1.

8. The fusion protein according to claim 1, wherein A is a scaffold protein.

9. The fusion protein according to claim 1, wherein M is an Fc part of an antibody.

10. The fusion protein according to claim 9, wherein said antibody is an IgG antibody.

11. The fusion protein according to claim 9, wherein said antibody is an IgG2 antibody.

12. The fusion protein according to claim 1, wherein M is an Fc part from an IgG2 antibody and A is extracellular Neprilysin.

13. The fusion protein according to claim 1, comprising an amino acid sequence according to SEQ ID NO. 11.

14. The fusion protein according to claim 1, wherein M is an Fc part from an IgG2 antibody and A is insulin-degrading enzyme.

15. The fusion protein according to claim 1, comprising an amino acid sequence according to SEQ ID NO. 12.

16. The fusion protein according to claim 1, wherein M is an Fc part from an IgG2 antibody and A is endothelin-converting enzyme 1.

17. The fusion protein according to claim 1, comprising an amino acid sequence according to SEQ ID NO. 13.

18. The fusion protein according to claim 1, wherein M is selected from pegylation and glycosylation.

19. The fusion protein according to claim 1, wherein M is a HSA.

20. The fusion protein according to claim 1, wherein M is a HSA binding domain.

21. The fusion protein according to claim 1, wherein M is a antibody binding domain.

22. The fusion protein according to claim 1, wherein M and A are linked together with a linker, L.

23. The fusion protein according to claim 22, wherein L is selected from a peptide and a chemical linker.

24. A method for reducing amyloid β peptide concentration, said method comprising administration of a fusion protein, according to claim 1.

25. A method according to claim 24, wherein reduction of amyloid β peptide is accomplished in plasma.

26. A method according to claim 24, wherein reduction of amyloid β peptide is accomplished in CSF.

27. A method according to claim 24, wherein reduction of amyloid β peptide is accomplished in CNS.

28. A pharmaceutical composition capable of degrading amyloid β peptide, comprising a pharmaceutically acceptable amount of fusion protein according to claim 1 together with a pharmaceutically acceptable carrier or excipient.

29. A method of prevention and/or treatment of a condition wherein degradation of amyloid β peptide is beneficial, comprising administering to a mammal, including man in need of such prevention and/or treatment, a therapeutically effective amount of a fusion protein according to claim 1.

30. A method of prevention and/or treatment of Alzheimer's disease, systemic amyloidosis or cerebral amyloid angiopathy, comprising administering to a mammal, including man in need of such prevention and/or treatment, a therapeutically effective amount of a fusion protein according to claim 1.

31-37. (canceled)
